(19) United States
(12) Patent Application Publication
(10) Pub. No.: US 2015/0057728 A1
(43) Pub. Date: Feb. 26, 2015

(54) EFFICIENT DYNAMIC STIMULATION IN AN IMPLANTED DEVICE

(71) Applicant: BIO CONTROL MEDICAL (B.C.M.) LTD., Yehud (IL)

(72) Inventors: Shai AYAL, Shoham (IL); Ehud COHEN, Ganei Tikva (IL)

(73) Assignee: BIO CONTROL MEDICAL (B.C.M.) LTD., Yehud (IL)

(21) Appl. No.: 14/528,665

(22) Filed: Oct. 30, 2014

Related U.S. Application Data
(63) Continuation-in-part of application No. 13/804,825, filed on Mar. 14, 2013.
Application No. 13/804,825 is a continuation of application No. 10/538,521, filed on Jan. 11, 2006, now Pat. No. 8,423,132.
Application No. 10/538,521 is a continuation-in-part of application No. 13/661,512, filed on Oct. 26, 2012, now Pat. No. 8,909,355.
Application No. 13/661,512 is a continuation of application No. 12/589,132, filed on Oct. 19, 2009, now Pat. No. 8,326,438.
Application No. 12/589,132 is a continuation of application No. 11/280,884, filed on Nov. 15, 2005, now Pat. No. 7,627,384.
Application No. 11/280,884 is a continuation-in-part of application No. 13/437,114, filed on Apr. 2, 2012, now Pat. No. 8,880,192.
(60) Provisional application No. 60/432,932, filed on Dec. 12, 2002.
(60) Provisional application No. 60/628,391, filed on Nov. 15, 2004.

(51) Int. Cl. A61N 1/05; A61N 1/375

(52) U.S. Cl. CPC A61N 1/0551; A61N 1/05; A61N 1/3756
USPC 607/116

(57) ABSTRACT

Apparatus is provided for applying current to a nerve of a subject, including a housing, adapted to be placed in a vicinity of the nerve; at least one cathode and at least one anode, fixed to the housing; a passive electrode, fixed to the housing; and a conducting element, which is electrically coupled to the passive electrode and is configured to extend to a remote location in a body of the subject at a distance of at least 1 cm from the housing. Other embodiments are also described.

### CROSS-REFERENCE TO RELATED APPLICATIONS

The present application is a continuation-in-part of:

(a) U.S. patent application Ser. No. 13/804,825, filed Mar. 14, 2013, which published as US Patent Application Publication 2013/0211480, which is a continuation of U.S. patent application Ser. No. 10/538,521, filed Jan. 11, 2006, now U.S. Pat. No. 8,423,132, which is the US national stage of International Application PCT/IL03/01062, filed Dec. 11, 2003, which published as PCT Publication WO 04/052444 and claims priority from U.S. Provisional Application 60/432,932, filed Dec. 12, 2002;

(b) U.S. patent application Ser. No. 13/661,512, filed Oct. 26, 2012, which published as US Patent Application Publication 2013/0053936, which is a continuation of U.S. patent application Ser. No. 12/589,132, filed Oct. 19, 2009, now U.S. Pat. No. 8,326,438, which is a continuation of U.S. patent application Ser. No. 11/280,884, filed Nov. 15, 2005, now U.S. Pat. No. 7,627,384, which claims the benefit of U.S. Provisional Application 60/628,391, filed Nov. 15, 2004; and

(c) U.S. patent application Ser. No. 13/437,114, filed Apr. 2, 2012, which published as US Patent Application Publication 2013/0261721.

All of the above-referenced applications are assigned to the assignee of the present application and are incorporated herein by reference.

### FIELD OF THE INVENTION

The present invention relates generally to electrical pulse generators, and specifically to pulse generators for electrical stimulation of tissue. The present invention also relates generally to electrical stimulation of tissue, and specifically to methods and devices for regulating the stimulation of nerves or other tissue.

### BACKGROUND OF THE INVENTION

Stimulation of tissue of a subject by applying an electrical potential to the tissue is well known in the medical art. Herein, in the specification and in the claims, tissue is to be understood as comprising muscle and/or nerve of a subject. Levels and type of stimulation used depend on a number of factors, such as whether the stimulation is applied externally, and the desired effect of the stimulation. When the tissue is stimulated directly, by one or more electrodes implanted in the tissue, levels of stimulation needed to achieve a specific desired effect are typically orders of magnitude less than the levels needed if the tissue is stimulated externally and/or indirectly. Devices for direct tissue stimulation, such as cardiac pacemakers, are typically implanted into the subject, and typically rely on an internal battery for producing their pulses.

Different types of pulses are known in the art for producing muscle stimulation. In the specification and in the claims, a biphasic pulse is assumed to be a pair of pulses having alternating positive and negative potentials, the biphasic pulse being able to stimulate the tissue; a mono-phase pulse is assumed to be a single uni-directional pulse which is able to stimulate the tissue; and alternating pulses are assumed to comprise a sequence of mono-phase pulses having alternating positive and negative potentials, each mono-phase pulse being able to stimulate the tissue. Typically, a time period between the pair of pulses comprising a biphasic pulse is of the order of 500 μs; a time period between sequential alternating pulses is of the order of 25 ms.

U.S. Pat. No. 5,391,191, to Holmstrom, whose disclosure is incorporated herein by reference, describes an implanted device for tissue stimulation. The device incorporates a current sensing mechanism which is applied to reduce tissue polarization, electrolysis effects, and detect tissue reaction. The device is implemented to deliver biphasic pulses, as well as a mono-phase pulses. Both types of pulses are used for stimulation; to reduce energy consumption, the device implements the mono-phase pulses.

It will be appreciated that efficiency in battery utilization in implanted devices is an important consideration, in order to increase battery life before recharging and/or replacement of the battery is required.

Anodal break excitation, also known as anode break excitation, may occur at an anode if tissue stimulation is abruptly halted. Such excitation is typically an undesired side effect of the tissue stimulation, and methods to reduce or eliminate the effect have been sought.

In the body, axons are grouped together for most of their lengths in nerve bundles. In a single bundle, many different axons travel together, branching only near their target organs. Important properties of natural axonal activity include that: (a) each axon can fire independently of its neighbors in the bundle, and (b) each axon conveys action potentials in only one direction, either afferently (towards the brain) or efferently (towards its target organ). These two properties, however, are not properties of the axons themselves. The axons are only active cables emanating from neurons which can trigger action potentials in them. Since each axon can be connected to a different neuron, they can fire independently. Also, because the axons are connected to a neuron only on one side, they only convey action potentials away from the neuron.

When axons are activated artificially by simple stimulation of a nerve bundle, both of these properties of natural axonal activity are lost: entire regions of the bundle are activated simultaneously, and the axons fire in both directions at once, since the action potential is not triggered at only one of the ends of the axons. The loss of these properties causes the effect of artificial stimulation to be less natural, and may result in side effects, because axons in the bundle in addition to the target axon are indiscriminately activated. To overcome these shortcomings of simple stimulation, two stimulation techniques have been developed: selective stimulation and unidirectional stimulation.

Selective electrical stimulation of nerve fibers is the activation of small fibers in a nerve bundle without the activation of the large fibers. This is advantageous, for example, when the target organ is innervated only by small fibers. In addition, stimulation of large fibers can cause unwanted side effects (see, for example, Rijkhoff et al. (1994) and Jones J F et al., cited hereinbelow). Often, in addition to selective stimulation, it is also advantageous to stimulate unidirectionally such that only organs at one end of the nerve receive signals.

As defined by Rattay, in the article, “Analysis of models for extracellular fiber stimulation,” IEEE Transactions on Biomedical Engineering, Vol. 36, no. 2, p. 676, 1989, which is incorporated herein by reference, the activation function (AF) of an unmyelinated axon is the second spatial derivative of the electric potential along an axon. In the region where the activation function is positive, the axon depolarizes, and in the region where the activation function is negative, the axon hyperpolarizes. If the activation function is sufficiently positive, then the depolarization will cause the axon to generate an action potential; similarly, if the activation function is sufficiently negative, then local blocking of action potentials transmission occurs. The activation function depends on the current applied, as well as the geometry of the electrodes and of the axon.

For a given electrode geometry, the equation governing the electrical potential is:

∇(σ∇U)=4πj,

where U is the potential, σ is the conductance tensor specifying the conductance of the various materials (electrode housing, axon, intracellular fluid, etc.), and j is a scalar function representing the current source density specifying the locations of current injection. The activation function is found by solving this partial differential equation for U. If an unmyelinated axon is defined to lie in the z direction, then the activation function is:

$$AF = \frac{{\partial }^{2} U}{\partial {z}^{2}} .$$

In a simple, illustrative example of a point electrode located a distance d from the axis of an axon in a uniformly-conducting medium with conductance σ, the two equations above are solvable analytically, to yield:

$$AF = \frac{{I}_{el}}{4 Πσ} \cdot \frac{2 {z}^{2} - {d}^{2}}{{( {z}^{2} + {d}^{2} )}^{2.5}} ,$$

where Iₑₗ is the electrode current. It is seen that when σ and d are held constant, and for a constant positive Iₑₗ (to correspond to anodal current), the minimum value of the activation function is negative, and is attained at z=0, i.e., at the point on the nerve closest to the source of the anodal current. Thus, the most negative point on the activation function corresponds to the place on a nerve where hyperpolarization is maximized, namely at the point on the nerve closest to the anode.

Additionally, this equation predicts positive “lobes” for the activation function on either side of z=0, these positive lobes peaking in their values at a distance which is dependent on each of the other parameters in the equation. The positive values of the activation function correspond to areas of depolarization, a phenomenon typically associated with cathodic current, not anodal current. However, it has been shown that excess anodal current does indeed cause the generation of action potentials adjacent to the point on a nerve corresponding to z=0, and this phenomenon is therefore called the “virtual cathode effect.” (An analogous, but reverse phenomenon, the “virtual anode effect” exists responsive to excess cathodic stimulation.)

The Rattay article also describes techniques for calculating the activation function for nerves containing myelinated axons. The activation function in this case varies as a function of the diameter of the axon in question. Thus, the activation function calculated for a 1 micron diameter myelinated axon is different from the activation function calculated for a 10 micron diameter axon.

U.S. Pat. No. 6,684,105 to Cohen et al., which is assigned to the assignee of the present application and is incorporated herein by reference, describes apparatus comprising an electrode device adapted to be coupled to longitudinal nervous tissue of a subject, and a control unit adapted to drive the electrode device to apply to the nervous tissue a current which is capable of inducing action potentials that propagate in the nervous tissue in a first direction, so as to treat a condition. The control unit is further adapted to suppress action potentials from propagating in the nervous tissue in a second direction opposite to the first direction.

U.S. Pat. No. 6,907,295 to Gross et al., which is assigned to the assignee of the present application and is incorporated herein by reference, describes apparatus for applying current to a nerve. A cathode is adapted to be placed in a vicinity of a cathodic longitudinal site of the nerve and to apply a cathodic current to the nerve. A primary inhibiting anode is adapted to be placed in a vicinity of a primary anodal longitudinal site of the nerve and to apply a primary anodal current to the nerve. A secondary inhibiting anode is adapted to be placed in a vicinity of a secondary anodal longitudinal site of the nerve and to apply a secondary anodal current to the nerve, the secondary anodal longitudinal site being closer to the primary anodal longitudinal site than to the cathodic longitudinal site.

A number of patents and articles describe methods and devices for stimulating nerves to achieve a desired effect. Often these techniques include a design for an electrode or electrode cuff.

U.S. Pat. No. 4,608,985 to Crish et al. and U.S. Pat. No. 4,649,936 to Ungar et al., which are incorporated herein by reference, describe electrode cuffs for selectively blocking orthodromic action potentials passing along a nerve trunk, in a manner intended to avoid causing nerve damage.

PCT Patent Publication WO 01/10375 to Felsen et al., which is incorporated herein by reference, describes apparatus for modifying the electrical behavior of nervous tissue. Electrical energy is applied with an electrode to a nerve in order to selectively inhibit propagation of an action potential.

U.S. Pat. No. 5,755,750 to Petruska et al., which is incorporated herein by reference, describes techniques for selectively blocking different size fibers of a nerve by applying direct electric current between an anode and a cathode that is larger than the anode.

U.S. Pat. No. 5,824,027 Hoffer et al., which is incorporated herein by reference, describes a nerve cuff having one or more sets of electrodes for selectively recording electrical activity in a nerve or for selectively stimulating regions of the nerve. Each set of electrodes is located in a longitudinally-extending chamber between a pair of longitudinal ridges which project into the bore of the nerve cuff. The ridges are electrically insulating and serve to improve the selectivity of the nerve cuff. The ridges seal against an outer surface of the nerve without penetrating the nerve. In an embodiment, circumferential end sealing ridges extend around the bore at each end of the longitudinal ridges, and are described as enhancing the electrical and/or fluid isolation between different ones of the longitudinally-extending chambers.

U.S. Pat. No. 4,628,942 to Sweeney et al., which is incorporated herein by reference, describes an annular electrode cuff positioned around a nerve trunk for imposing electrical signals on to the nerve trunk for the purpose of generating unidirectionally propagated action potentials. The electrode cuff includes an annular cathode having a circular passage therethrough of a first diameter. An annular anode has a larger circular passage therethrough of a second diameter, which second diameter is about 1.2 to 3.0 times the first diameter. A non-conductive sheath extends around the anode, cathode, and nerve trunk. The anode and cathode are placed asymmetrically to one side of the non-conductive sheath.

U.S. Pat. No. 5,423,872 to Cigaina, which is incorporated herein by reference, describes a process for treating obesity and syndromes related to motor disorders of the stomach of a patient. The process consists of artificially altering, by means of sequential electrical pulses and for preset periods of time, the natural gastric motility of the patient to prevent emptying or to slow down gastric transit. The '872 patent describes an electrocatheter adapted to be coupled to a portion of the stomach. A portion of the electrocatheter has a rough surface for producing a fibrous reaction of the gastric serosa, in order to contribute to the firmness of the anchoring.

U.S. Pat. No. 4,573,481 to Bullara, which is incorporated herein by reference, describes an implantable helical electrode assembly, configured to fit around a nerve, for electrically triggering or measuring an action potential or for blocking conduction in nerve tissue. A tissue-contacting surface of each electrode is roughened to increase the electrode surface area.

The following patents, which are incorporated herein by reference, may be of interest:
U.S. Pat. No. 6,230,061 to Hartung
U.S. Pat. No. 5,282,468 to Klepinski
U.S. Pat. No. 4,535,785 to van den Honert et al.
U.S. Pat. No. 5,215,086 to Terry et al.
U.S. Pat. No. 6,341,236 to Osorio et al.
U.S. Pat. No. 5,487,756 to Kallesoe et al.
U.S. Pat. No. 5,634,462 to Tyler et al.
U.S. Pat. No. 6,456,866 to Tyler et al.
U.S. Pat. No. 4,602,624 to Naples et al.
U.S. Pat. No. 6,600,956 to Maschino et al.
U.S. Pat. No. 5,199,430 to Fang et al.

The following articles, which are incorporated herein by reference, may be of interest:
Ungar IJ et al., “Generation of unidirectionally propagating action potentials using a monopolar electrode cuff,” Annals of Biomedical Engineering, 14:437-450 (1986)
Sweeney JD et al., “An asymmetric two electrode cuff for generation of unidirectionally propagated action potentials,” IEEE Transactions on Biomedical Engineering, vol. BME-33(6) (1986)
Sweeney JD et al., “A nerve cuff technique for selective excitation of peripheral nerve trunk regions,” IEEE Transactions on Biomedical Engineering, 37(7) (1990)
Naples GG et al., “A spiral nerve cuff electrode for peripheral nerve stimulation,” by IEEE Transactions on Biomedical Engineering, 35(11) (1988)
van den Honert C et al., “Generation of unidirectionally propagated action potentials in a peripheral nerve by brief stimuli,” Science, 206:1311-1312 (1979)
van den Honert C et al., “A technique for collision block of peripheral nerve: Single stimulus analysis,” MP-11, IEEE Trans. Biomed. Eng. 28:373-378 (1981)
van den Honert C et al., “A technique for collision block of peripheral nerve: Frequency dependence,” MP-12, IEEE Trans. Biomed. Eng. 28:379-382 (1981)
Rijkhoff N J et al., “Acute animal studies on the use of anodal block to reduce urethral resistance in sacral root stimulation,” IEEE Transactions on Rehabilitation Engineering, 2(2):92-99 (1994)
Mushahwar VK et al., “Muscle recruitment through electrical stimulation of the lumbo-sacral spinal cord,” IEEE Trans Rehabil Eng, 8(1):22-9 (2000)
Deurloo KE et al., “Transverse tripolar stimulation of peripheral nerve: a modelling study of spatial selectivity,” Med Biol Eng Comput, 36(1):66-74 (1998)
Tarver WB et al., “Clinical experience with a helical bipolar stimulating lead,” Pace, Vol. 15, October, Part II (1992)
Hoffer JA et al., “How to use nerve cuffs to stimulate, record or modulate neural activity,” in Neural Prostheses for Restoration of Sensory and Motor Function, Chapin JK et al. (Eds.), CRC Press (1st edition, 2000)
Jones J F et al., “Heart rate responses to selective stimulation of cardiac vagal C fibres in anaesthetized cats, rats and rabbits,” J Physiol 489(Pt 1):203-14 (1995)
Evans M S et al., “Intraoperative human vagus nerve compound action potentials,” Acta Neurol Scand 110:232-238 (2004)

In physiological muscle contraction, nerve fibers are recruited in the order of increasing size, from smaller-diameter fibers to progressively larger-diameter fibers. In contrast, artificial electrical stimulation of nerves using standard techniques recruits fibers in a larger- to smaller-diameter order, because larger-diameter fibers have a lower excitation threshold. This unnatural recruitment order causes muscle fatigue and poor force gradation. Techniques have been explored to mimic the natural order of recruitment when performing artificial stimulation of nerves to stimulate muscles.

Fitzpatrick et al., in “A nerve cuff design for the selective activation and blocking of myelinated nerve fibers,” Ann. Conf. of the IEEE Eng. in Medicine and Biology Soc, 13(2), 906 (1991), which is incorporated herein by reference, describe a tripolar electrode used for muscle control. The electrode includes a central cathode flanked on its opposite sides by two anodes. The central cathode generates action potentials in the motor nerve fiber by cathodic stimulation. One of the anodes produces a complete anodal block in one direction so that the action potential produced by the cathode is unidirectional. The other anode produces a selective anodal block to permit passage of the action potential in the opposite direction through selected motor nerve fibers to produce the desired muscle stimulation or suppression.

The following articles, which are incorporated herein by reference, may be of interest:
Rijkhoff N J et al., “Orderly recruitment of motoneurons in an acute rabbit model,” Ann. Conf. of the IEEE Eng., Medicine and Biology Soc., 20(5):2564 (1998)
Rijkhoff N J et al., “Selective stimulation of small diameter nerve fibers in a mixed bundle,” Proceedings of the Annual Project Meeting Sensations/Neuros and Mid-Term Review Meeting on the TMR-Network Neuros, Apr. 21-23, 1999, pp. 20-21 (1999)
Baratta R et al., “Orderly stimulation of skeletal muscle motor units with tripolar nerve cuff electrode,” IEEE Transactions on Biomedical Engineering, 36(8):836-43 (1989)

The following articles, which are incorporated herein by reference, describe techniques using cuff electrodes to selectively excite peripheral nerve fibers distant from an electrode without exciting nerve fibers close to the electrode:
Grill WM et al., “Inversion of the current-distance relationship by transient depolarization,” IEEE Trans Biomed Eng, 44(1):1-9 (1997)
Goodall EV et al., “Position-selective activation of peripheral nerve fibers with a cuff electrode,” IEEE Trans Biomed Eng, 43(8):851-6 (1996)
Veraart C et al., “Selective control of muscle activation with a multipolar nerve cuff electrode,” IEEE Trans Biomed Eng, 40(7):640-53 (1993)

One method used for selective stimulation is based on the observation that the stimulation/block threshold of fibers is inversely proportional to their radius. Thus, to stimulate only small fibers, all fibers are stimulated using a large cathodic current, and the large fibers are then blocked using a smaller anodal current, the net effect being action potential propagation in the small fibers only. To achieve unidirectional stimulation, one uses larger anodic currents on one side, thus blocking all fibers on that side. Because of the intrinsic physiological timescales of the ion channels in the axon, to block an action potential one uses a long pulse of approximately 1 millisecond. This long pulse may degrade stimulation efficiency. By comparison, an action potential can be triggered with pulses as short as approximately 10 microseconds.

A method for selective stimulation is described in Lertmanorat Z et al., “A novel electrode array for diameter-dependent control of axonal excitability: a simulation study,” IEEE Transactions on Biomedical Engineering 51(7):1242-1250 (2004), which is incorporated herein by reference. The described Electrode Array Selective Stimulation (EASS) method relies on the structure of myelinated fibers and employs electrode arrays. The myelinated fibers are surrounded by a sheath of myelin, which functions as an isolator. In this sheath there are gaps at regular intervals, called nodes of Ranvier. The gap distance is roughly proportional to the radius of the axon. Ion channels are present only at these gaps.

The principle of EASS is that if an electric field is produced which is periodic along a nerve, and the period matches the gap distance of an axon with a certain diameter, then the axon essentially “sees” a constant electric field, so that no stimulation/block occurs. Axons of different gap-distances see a varying field and are thus stimulated/blocked. The variation in the electric field that an axon “sees” depends on the ratio between its gap distance and the field period. The variation also depends on the radial distance (depth) from the electrode to the axon. As the axon gets further away from the electrode, the field becomes less varying since the cathodic and anodal fields tend to cancel each other. The inventors of the present patent application estimate that the fields vary in a substantial manner up to a radial distance of about one period of the field. It should be noted that at all distances, the field has the same periodicity. Therefore, axons with a nodal gap distance which matches the field period will not be activated at any depth, but other axons may not be activated because the field becomes too weak.

Since the gap distance is proportional to the axon radius, by selecting a period for the field to change, a range of axon radii can be selected which are substantially not affected by the electric field. Setting the period of the field to be the gap distance of large fibers ensures that large fibers will not be affected by the stimulation. An advantage of this method for selective stimulation is that stimulus duration can be short; no blocking is needed since the large fibers are simply not activated.

An EASS electrode can be made by placing an alternating series of anode and cathodes along the axon, spaced a gap width apart. The cathodes and anodes can be ring shaped to give better field uniformity inside the nerve.

The main shortcoming of this method is that while it enables selective stimulation with short pulses, it does not provide unidirectional stimulation.

A number of patents and articles describe methods and devices for stimulating nerves to achieve a desired effect. Often these techniques include a design for an electrode or electrode cuff.

### SUMMARY OF THE INVENTION

It is an object of some aspects of the present invention to provide a method and apparatus for stimulating tissue of a subject.

It is a further object of some aspects of the present invention to reduce or eliminate anodal break excitation.

In a preferred embodiment of the present invention, a stimulation device comprises charging circuitry for charging a stimulation capacitor to an operating voltage level. Switching circuitry in the device is coupled to the charging circuitry. The switching circuitry generates both biphasic and alternating pulses, herein termed stimulation pulses, from uni-directional pulses which are generated by discharging the stimulation capacitor from the operating voltage level for a pulse period. The stimulation pulses are delivered to tissue of a subject by electrodes implanted therein, generating a tissue stimulation level which is a function of the operating voltage and the pulse period. Initially both the operating voltage and the pulse period are preset by an operator of the device, so as to achieve a desired tissue stimulation level. During operation of the device, a potential is measured on the stimulation capacitor at discharge, so as to measure an impedance of the tissue. Responsive to the impedance, the operating voltage level and/or the preset pulse period are adjusted in order to maintain the tissue stimulation level at the desired tissue stimulation level.

The charging circuitry is most preferably driven by an internal battery contained in the device. The charging circuitry, operated by a micro-controller, comprises a direct current (DC) charging circuit and an alternating current (AC) charging circuit which are each able to charge the stimulation capacitor by respective differential potentials. The micro-controller is able to minimize battery energy dissipation by using either or both circuits, and to set the time of use of each circuit in order to charge the stimulation capacitor to the initial operating voltage level. The AC circuit enables the operating voltage to be reached regardless of a voltage delivered by the battery. Similarly, for any adjusted operating voltage or other pulse parameter such as the pulse period, the micro-controller sets the time of use of each circuit so as to charge the stimulation capacitor to the adjusted operating voltage efficiently.

Parameters such as tissue impedance and operating voltage level are derived from measured potentials on the stimulation capacitor as it discharges. Thus, no current sensing mechanism, as is used by other tissue stimulators known in the art, is required in preferred embodiments of the present invention. Current sensing mechanisms, typically resistors, drain energy. Not implementing such a mechanism achieves a significant saving in energy supplied by a battery powering the stimulation device.

In some preferred embodiments of the present invention, a resistive element having a controlled resistance is coupled between the electrodes so as to be able to short-circuit an interface capacitance formed in the tissue being stimulated. The resistive element preferably comprises a field effect transistor (FET) having a gate which acts as a control electrode for the resistive element, the gate being activated by a control signal from the micro-controller. By short-circuiting the electrodes and the interface capacitance at times chosen by the micro-controller, electrolysis effects at the electrodes may be reduced and even eliminated, and the tissue stimulation level may be more precisely defined.

In preferred embodiments of the present invention, the micro-controller comprises an analog-to-digital converter (ADC). Most preferably, the stimulation device comprises a calibration circuit which generates a DC reference voltage which is substantially invariant even with variation of battery voltage from a battery powering the device. A memory in the micro-controller comprises a look-up table which has a one-to-one mapping between a digital value generated by the ADC responsive to the reference voltage and the battery voltage at which the ADC is operating. The look-up table also comprises a one-to-one mapping between the digital value and a multiplicative correction factor. The micro-controller multiplies other digital values, generated as the ADC is measuring voltages within the stimulation device, in order to adjust the other digital values due to changes in the battery voltage.

In an alternative preferred embodiment of the present invention, the charging circuitry is coupled to current controlled stimulation circuitry, rather than to the switching circuitry. The current controlled stimulation circuitry is configured to apply a pre-determined voltage-time relationship to the tissue. The voltage-time relationship is preferably substantially linear, and is applied to discharge the interface capacitance of the tissue being stimulated. By discharging the interface capacitance in a linear manner with a current limiter, anodal break excitation of the tissue is substantially eliminated.

There is therefore provided, according to a preferred embodiment of the present invention, a method for measuring impedance of a tissue, including:

charging a capacitor to a potential;

discharging the capacitor for a discharge period through the tissue;

measuring a voltage drop on the capacitor over the discharge period; and

determining the impedance of the tissue responsive to the potential, the voltage drop, and the discharge period.

Preferably, charging the capacitor includes:

providing a first circuit which is adapted to charge the capacitor to a first voltage;

providing a second circuit which is adapted to charge the capacitor to a second voltage;

measuring the potential on the capacitor;

determining a first charging period for the first circuit and a second charging period for the second circuit, responsive to the potential, so that the first and second charging periods substantially total to the predetermined period and so that the first and second voltages substantially total to the predetermined differential potential; and

operating the first circuit for the first charging period and the second circuit for the second charging period, the circuits being operated sequentially.

The first circuit preferably includes a resistive element through which the capacitor is charged by a substantially direct current (DC), and the second circuit preferably includes an inductor, a switching element, and a diode, which are operative to generate a substantially alternating current (AC) and to rectify the AC so as to charge the capacitor.

There is further provided, according to a preferred embodiment of the present invention, a method for stimulating a tissue, including:

charging a capacitor to a first potential;

discharging the capacitor for a first discharge period through the tissue;

measuring a voltage drop on the capacitor over the first discharge period;

determining an impedance of the tissue responsive to the first potential, the voltage drop, and the first discharge period;

determining a second potential and a second discharge period, responsive to the impedance and a predetermined desired tissue stimulation level;

charging the capacitor to the second potential; and

discharging the capacitor for the second discharge period through the tissue.

Preferably, discharging the capacitor for the first discharge period and discharging the capacitor for the second discharge period each include discharging alternating pulses through the tissue, each alternating pulse including a positive-going pulse followed by a negative-going pulse, so that a time between the positive-going pulse and the negative-going pulse is substantially equal to half a period of the alternating pulses.

Discharging the capacitor for the first discharge period and discharging the capacitor for the second discharge period each preferably include discharging biphasic pulses through the tissue, each biphasic pulse including a positive-going pulse followed by a negative-going pulse, so that a time between the positive-going pulse and the negative-going pulse is substantially less than half a period of the biphasic pulses.

Further preferably, discharging the biphasic pulses includes discharging a first biphasic pulse including a first positive-going pulse followed by a first negative-going pulse, followed by a second biphasic pulse including a second negative-going pulse followed by a second positive-going pulse.

Preferably, discharging the capacitor for the first discharge period and discharging the capacitor for the second discharge period each include discharging the capacitor responsive to a control signal generated by the tissue.

The second discharge period is preferably subsequent to the first discharge period.

There is further provided, according to a preferred embodiment of the present invention, apparatus for measuring impedance of a tissue, including:

a capacitor; and

circuitry which is adapted to:

charge the capacitor to a potential,

discharge the capacitor for a discharge period through the tissue,

measure a voltage drop on the capacitor over the discharge period, and

determine the impedance of the tissue responsive to the potential, the voltage drop, and the discharge period.

Preferably, the circuitry includes:

a first circuit which is adapted to charge the capacitor to a first voltage;

a second circuit which is adapted to charge the capacitor to a second voltage;

and wherein the circuitry is further adapted to measure the potential on the capacitor,

determine a first charging period for the first circuit and a second charging period for the second circuit, responsive to the potential, so that the first and second charging periods substantially total to the predetermined period and so that the first and second voltages substantially total to the predetermined differential potential, and

operate the first circuit for the first charging period and the second circuit for the second charging period, the circuits being operated sequentially.

The first circuit preferably includes a resistive element through which the capacitor is charged by a substantially direct current (DC), and the second circuit preferably includes an inductor, a switching element, and a diode, which are operative to generate a substantially alternating current (AC) and to rectify the AC so as to charge the capacitor.

There is further provided, according to a preferred embodiment of the present invention, apparatus for changing a potential across a capacitor by a predetermined differential potential in a predetermined time period, including:

a first circuit which is adapted to charge the capacitor to a first voltage;

a second circuit which is adapted to charge the capacitor to a second voltage; and

a controller which measures the potential on the capacitor, and responsive thereto and to the predetermined differential potential and the predetermined time period operates the first circuit and the second circuit sequentially for respective periods of time substantially totaling the predetermined time period so as to charge the capacitor by the predetermined differential potential substantially totaling the first and the second voltages.

The apparatus preferably includes a memory wherein is stored a first charging rate for the first circuit and a second charging rate for the second circuit, and wherein the controller is adapted to determine the respective periods of time responsive to the first and the second charging rates.

Preferably, the first circuit dissipates a first energy to charge the capacitor to the first voltage and the second circuit dissipates a second energy to charge the capacitor to the second voltage, and the controller is adapted to determine the respective periods of time responsive to the first and the second energies.

The controller is preferably adapted to determine the respective periods so that a sum of the first and the second energies is a minimum.

Preferably, the first circuit includes a resistive element through which the capacitor is charged by a substantially direct current (DC), and the second circuit includes an inductor, a switching element, and a diode, which are operative to generate a substantially alternating current (AC) and to rectify the AC so as to charge the capacitor.

The apparatus preferably includes a battery supplying a battery voltage, wherein the first circuit includes a resistive element through which the capacitor is charged by a substantially direct current (DC), and wherein the second circuit includes an inductor, a switching element, and a diode, which are operative to generate a substantially alternating current (AC) and to rectify the AC so as to charge the capacitor, wherein the first voltage is a predetermined fraction, greater than 0 and less than 1, of the battery voltage, and wherein the predetermined differential potential is greater than the battery voltage.

There is further provided, according to a preferred embodiment of the present invention, apparatus for stimulating a tissue having a tissue capacitance, including:

a capacitor;

circuitry which is adapted to:

charge the capacitor to a potential,

discharge the capacitor for a discharge period through the tissue; and

a resistive element, having a resistance which is controlled by the circuitry, and which is coupled to the circuitry and which is adapted to substantially short-circuit the tissue capacitance responsive to a control signal generated by the circuitry.

Preferably, the circuitry is adapted to generate the control signal at a time so as to implement a predetermined stimulation level to the tissue, and the time preferably directly follows a completion of the discharge period.

Preferably, the resistance includes a value so that substantially no anodal break excitation occurs in the tissue.

There is further provided, according to a preferred embodiment of the present invention, apparatus for measuring a voltage, including:

a battery which supplies a direct current (DC) voltage;

a DC voltage reference source, which generates a substantially invariant reference voltage, and which is powered by the battery;

an analog-to-digital converter (ADC) which generates a digital value responsive to receiving the reference voltage as an analog input, and which is powered by the battery;

a memory, comprising an ADC look-up table having a one-to-one mapping between the digital value and the DC voltage; and

a processor, which is adapted to use the ADC look-up table to determine the DC voltage responsive to the digital value.

Preferably, the ADC look-up table includes a further one-to-one mapping between the digital value and a multiplicative correction factor which is operative to multiply the digital value so as to generate an improved digital value, and the ADC is adapted to receive an alternative DC voltage and to generate an alternative digital value responsive thereto, and the processor is adapted to determine the alternative DC voltage responsive to the alternative digital value and the multiplicative correction factor.

The apparatus preferably includes a plurality of resistors acting as a voltage divider which generate the alternative DC voltage, and one of the resistors preferably includes an internal resistance of the ADC.

There is further provided, according to a preferred embodiment of the present invention, a method for changing a potential across a capacitor by a predetermined differential potential in a predetermined time period, including:

providing a first circuit which is adapted to charge the capacitor to a first voltage;

providing a second circuit which is adapted to charge the capacitor to a second voltage;

measuring a potential on the capacitor;

determining a first charging period for the first circuit and a second charging period for the second circuit, responsive to the potential, so that the first and second charging periods substantially total to the predetermined period and so that the first and second voltages substantially total to the predetermined differential potential; and

operating the first circuit for the first charging period and the second circuit for the second charging period, the circuits being operated sequentially.

The method preferably includes storing a first charging rate for the first circuit and a second charging rate for the second circuit in a memory, wherein determining the first charging period and the second charging period includes determining the charging periods responsive to the first and the second charging rates.

Preferably, the first circuit dissipates a first energy to charge the capacitor to the first voltage and the second circuit dissipates a second energy to charge the capacitor to the second voltage, and wherein determining the first charging period and the second charging period includes determining the charging periods responsive to the first and the second energies.

Preferably, determining the charging periods includes determining the charging periods so that a sum of the first and the second energies is a minimum.

Further preferably, the first circuit includes a resistive element through which the capacitor is charged by a substantially direct current (DC), and the second circuit includes an inductor, a switching element, and a diode, which are operative to generate a substantially alternating current (AC) and to rectify the AC so as to charge the capacitor.

The method preferably includes providing a battery that supplies a battery voltage, wherein the first circuit includes a resistive element through which the capacitor is charged by a substantially direct current (DC), and wherein the second circuit includes an inductor, a switching element, and a diode, which are operative to generate a substantially alternating current (AC) and to rectify the AC so as to charge the capacitor, wherein the first voltage is a predetermined fraction, greater than 0 and less than 1, of the battery voltage, and wherein the predetermined differential potential is greater than the battery voltage.

There is further provided, according to a preferred embodiment of the present invention, apparatus for stimulating tissue having a capacitance, including:

charge circuitry which is adapted to apply a potential to the tissue, causing a voltage to develop across the capacitance of the tissue; and

discharge circuitry which is adapted to inject a current to the tissue so as to discharge the capacitance, the current being substantially independent of the voltage across the capacitance.

Preferably, the charge circuitry includes a stimulation capacitor, an inductor, and a micro-controller which is adapted to apply pulses having a variable duty cycle to the inductor, and wherein the micro-controller causes the inductor to charge the stimulation capacitor to the voltage by altering the variable duty cycle.

Preferably, the current is substantially fixed.

Preferably, the potential causes a stimulation current in the tissue, and the current injected by the discharge circuitry is preferably a substantially pre-set fraction of the stimulation current.

The current preferably includes a value that substantially eliminates anodal break excitation of the tissue, the value is preferably less than a pre-set fraction of a stimulation current caused by the potential, and the pre-set fraction is preferably approximately 5%.

The discharge circuitry is preferably adapted to measure the voltage across the capacitance, and is preferably adapted to halt injection of the current to the tissue when the voltage is substantially zero.

The apparatus preferably includes:

a battery having a first battery terminal and a second battery terminal coupled to ground and generating a battery voltage which powers at least a first part of the charge circuitry and at least a second part of the discharge circuitry; and

a first and a second stimulation electrode between which the capacitance is formed,

wherein the first battery terminal and the first stimulation electrode are connected, and wherein the charge circuitry generates the potential between the first and the second stimulation electrodes, and wherein the discharge circuitry injects the current between the first and the second stimulation electrodes.

The apparatus preferably also includes:

a stimulation capacitor which receives a stimulation potential generated by the charge circuitry; and

a detector which monitors a second-stimulation-electrode potential on the second stimulation electrode, the detector being coupled between ground and the stimulation potential.

Further preferably, the apparatus includes a micro-controller which receives a Boolean signal from the detector in response to the second-stimulation-electrode potential, and which decrements a targeted voltage set by the micro-controller in response to the signal being true, and which increments the targeted voltage in response to the signal being false.

The apparatus preferably includes a micro-controller which is adapted to measure a time to discharge the capacitance, and to generate a measure of the capacitance in response to the time.

The apparatus preferably includes a micro-controller which is adapted to measure a time to apply the potential to the tissue, and to generate a measure of the capacitance in response to the time.

The apparatus preferably further includes:

a detector which monitors a state of at least part of the charge circuitry, and which generates a state signal in response to the state; and

a micro-controller which receives the state signal and which sets the potential in response thereto.

Preferably, the micro-controller generates a pulse, at the potential, in a sequence of pulses and sets a target voltage in response to the state signal and the potential, and wherein the charge circuitry is adapted to alter the potential to a future potential in response to the target voltage, and to apply the future potential to a subsequent pulse in the sequence.

Further preferably, the charge circuitry is adapted to measure an impedance of the tissue, and to alter the potential applied to the tissue in response to the impedance.

There is further provided, according to a preferred embodiment of the present invention, a method for stimulating tissue having a capacitance, including:

applying a potential to the tissue so as to cause a voltage to develop across the capacitance of the tissue; and

injecting a current to the tissue so as to discharge the capacitance, the current being substantially independent of the voltage across the capacitance.

Preferably, the current is substantially fixed.

Preferably, the potential causes a stimulation current in the tissue, and the current injected is a substantially pre-set fraction of the stimulation current, and the current preferably includes a value that substantially eliminates anodal break excitation of the tissue, and the value is preferably less than a pre-set fraction of a stimulation current caused by the potential, and the pre-set fraction is preferably approximately 5%.

The method preferably includes measuring the voltage across the capacitance, and halting injection of the current to the tissue when the voltage is substantially zero.

The method preferably includes:

providing a battery having a first battery terminal and a second battery terminal coupled to ground;

providing a first and a second stimulation electrode between which the capacitance is formed;

connecting the first battery terminal and the first stimulation electrode;

generating the potential between the first and the second stimulation electrodes;

injecting the current between the first and the second stimulation electrodes;

providing a stimulation capacitor which receives a stimulation potential in response to applying the potential;

coupling a detector between ground and the stimulation potential; and

monitoring with the detector a second-stimulation-electrode potential on the second stimulation electrode.

The method preferably includes:

receiving a Boolean signal from the detector in response to the second-stimulation-electrode potential;

setting a targeted voltage;

decrementing the targeted voltage in response to the signal being true; and

incrementing the targeted voltage in response to the signal being false.

The method preferably includes measuring a time to discharge the capacitance, and generating a measure of the capacitance in response to the time.

The method preferably includes measuring a time to apply the potential to the tissue, and generating a measure of the capacitance in response to the time.

The method preferably includes:

monitoring a state of charge circuitry adapted to apply the potential, and generating a state signal in response to the state; and

receiving the state signal and setting the potential in response thereto.

The method preferably includes:

generating a pulse, at the potential, in a sequence of pulses and setting a target voltage in response to the state signal and the potential;

altering the potential to a future potential in response to the target voltage; and

applying the future potential to a subsequent pulse in the sequence.

The method preferably includes measuring an impedance of the tissue, and altering the potential applied to the tissue in response to the impedance.

There is further provided, according to a preferred embodiment of the present invention, apparatus for stimulating tissue having a capacitance, including:

charge circuitry which is adapted to apply a potential to the tissue, causing a voltage to develop across the capacitance of the tissue;

discharge circuitry which is adapted to inject a current to the tissue so as to discharge the capacitance; and

feedback circuitry which is adapted to monitor the potential and to control the current in response to the potential.

There is further provided, according to a preferred embodiment of the present invention, a method for stimulating tissue having a capacitance, including:

applying a potential to the tissue so as to cause a voltage to develop across the capacitance of the tissue;

injecting a current to the tissue so as to discharge the capacitance;

monitoring the potential to generate a monitored potential; and

controlling the current in response to the monitored potential.

In some embodiments of the present invention, an electrode assembly for applying current to a nerve comprises at least one cathode, at least one anode, and two or more passive electrodes, which are fixed within a housing. The electrode assembly comprises a conducting element, typically a wire, which electrically couples the passive electrodes to one another. A “passive electrode,” as used in the present application including the claims, is an electrode that is electrically “device-coupled” to neither (a) any circuitry that is electrically device-coupled to the at least one cathode or the at least one anode, nor (b) an energy source. “Device-coupled” means coupled, directly or indirectly, by components of a device, and excludes coupling via tissue of a subject. (It is noted that the passive electrodes may be passive because of a software-controlled setting of the electrode assembly, and that the software may intermittently change the setting such that these electrodes are not passive.) To “passively electrically couple,” as used in the present application including the claims, means to couple using at least one passive electrode and no non-passive electrodes. The passive electrodes and conducting element create an electrical path for current that would otherwise leak outside the electrode assembly and travel around the outside of the housing through tissue of the subject.

For some applications, the at least one cathode and at least one anode are positioned within the housing longitudinally between the two or more passive electrodes. Alternatively, at least one of the passive electrodes is positioned between the at least one cathode and the at least one anode. For some applications, the electrode assembly is configured to apply unidirectional stimulation to the nerve. Alternatively or additionally, the electrode assembly is configured to selectively stimulate fibers of the nerve having certain diameters.

In some embodiments of the present invention, an electrode assembly for applying current to a nerve comprises two cathodes and at least one anode, which are fixed within a housing such that no anodes are positioned between the two cathodes. (If any anode is positioned between the two cathodes, then in at least one mode of operation, this anode applies no more than a trivial amount of anodal current to the nerve.) Typically, a distance between the two cathodes is equal to at least a radius of the nerve, e.g., at least 1.5 times the radius of the nerve. This electrode configuration creates a combined cathode having an activation function a peak of which has a magnitude less that of the anode, which results in unidirectional stimulation of the nerve in the direction of the cathodes. Typically, this electrode configuration also creates a virtual anode on the side of the cathodes opposite that of the anode, which results in selective fiber stimulation of fibers of the nerve having relatively small diameters. For some applications, the electrode assembly additionally comprises two or more passive electrodes coupled to one another, as described above, positioned such that the cathodes and the at least one anode are between the passive electrodes.

In some embodiments of the present invention, an electrode assembly for applying current to a nerve comprises a housing, which is placed in a vicinity of the nerve, one or more electrodes, fixed to the housing, and two longitudinally-elongated end insulating elements, fixed to the housing such that all of the electrodes are longitudinally between the insulating elements. Each of the end insulating elements has a length of at least 2 mm, such as at least 3 mm, or at least 4 mm. This elongation of the end insulating elements tends to lengthen the electrical path around the outside of the electrode assembly through tissue of the subject, thereby reducing the current that leaks from the assembly and flows through this path.

There is therefore provided, in accordance with an embodiment of the present invention, apparatus for applying current to a nerve, including:

a housing, adapted to be placed in a vicinity of the nerve;

at least one cathode and at least one anode, fixed to the housing;

two or more passive electrodes, fixed to the housing; and

a conducting element, which electrically couples the passive electrodes to one another.

In an embodiment, the two or more passive electrodes include exactly two passive electrodes.

In an embodiment, the at least one cathode and the at least one anode are fixed longitudinally between the two or more passive electrodes. Alternatively, at least one of the passive electrodes is fixed longitudinally between the at least one cathode and the at least one anode.

For some applications, the at least one anode includes one or more anodes which are configured to apply to the nerve an inhibiting current capable of inhibiting cathode-induced action potentials traveling in a non-therapeutic direction in the nerve. For some applications, the at least one cathode includes one or more cathodes which are configured to apply to the nerve a stimulating current, which is capable of inducing action potentials in a first set and a second set of nerve fibers of the nerve, and the at least one anode includes one or more anodes that are configured to apply to the nerve an inhibiting current, which is capable of inhibiting the induced action potentials in the second set of nerve fibers, the nerve fibers in the second set having generally larger diameters than the nerve fibers in the first set.

For some applications, the electrodes include ring electrodes. For some applications, the conducting element includes at least one passive element that impedes passage of current through the conducting element. For some applications, the housing includes one or more insulating elements that separate one or more of the at least one cathode, the at least one anode, and the passive electrodes, the insulating elements positioned closer to the nerve than are the at least one cathode, the at least one anode, and the passive electrodes.

There is also provided, in accordance with an embodiment of the present invention, apparatus for applying current to a nerve of a subject, including:

a housing, adapted to be placed in a vicinity of the nerve;

at least one cathode and at least one anode, fixed to the housing;

a passive electrode, fixed to the housing; and

a conducting element, which is electrically coupled to the passive electrode and is configured to extend to a remote location in a body of the subject at a distance of at least 1 cm from the housing, such as at least 2 cm, e.g., at least 3 cm.

For some applications, the apparatus further includes a remote electrode, a first end of the conducting element is coupled to the passive electrode, and a second end of the conducting element is coupled to the remote electrode. For some applications, the remote electrode is configured for insertion into muscle tissue of the subject.

There is further provided, in accordance with an embodiment of the present invention, apparatus for applying current to a nerve having a radius, including:

a housing, adapted to be placed in a vicinity of the nerve; and

two or more cathodes and one or more anodes, fixed to the housing such that no anodes are positioned longitudinally between the two or more cathodes.

In an embodiment, the apparatus includes two or more passive electrodes, fixed to the housing, such that the cathodes and the anodes are longitudinally between the passive electrodes; and a conducting element, which electrically couples the passive electrodes to one another.

For some applications, the two or more cathodes and the one or more anodes include ring electrodes. For some applications, the housing includes one or more insulating elements that separate one or more of the cathodes and the anodes, the insulating elements positioned closer to the nerve than are the cathodes and the anodes.

In an embodiment, the cathodes and anodes are fixed to the housing such that no cathodes are positioned longitudinally between the one or more anodes.

In an embodiment, the two or more cathodes are fixed to the housing at respective cathodic longitudinal locations, and are configured to apply to the nerve a stimulating current, which is capable of inducing action potentials in a first set and a second set of nerve fibers of the nerve; the one or more anodes are fixed to the housing at respective anodal locations, such that no cathodes are positioned longitudinally between the one or more anodes; the cathodes are configured to produce a virtual anode effect at a virtual anodal longitudinal site of the nerve, which is capable of inhibiting the induced action potentials in the second set of nerve fibers, the nerve fibers in the second set having generally larger diameters than the nerve fibers in the first set; and the cathodic locations are between (a) the anodal locations and (b) the virtual anodal site.

In an embodiment, the two or more cathodes are fixed to the housing at respective cathodic locations; the one or more anodes include a single anode, fixed to the housing at an anodal location; the anode is configured to produce a virtual cathode effect at a virtual cathodic longitudinal site of the nerve, which is incapable of generating action potentials in the nerve; and the anodal location is between (a) the cathodic locations and (b) the virtual cathodic site. Alternatively, the anode is configured to produce the virtual cathode effect at the virtual cathodic longitudinal site which does not generate action potentials in more than 10% of axons in the nerve.

In an embodiment, the cathodes are positioned such that a closest distance between two of the two or more cathodes is equal to at least the radius of the nerve, such as equal to at least 1.5 times the radius of the nerve.

In an embodiment, the cathodes are configured to apply respective cathodic currents to the nerve; the anodes are configured to apply respective anodal currents to the nerve; and for any given depth within the nerve, for a myelinated axon within the nerve of diameter less than 10 microns, the cathodic currents define, in combination, for the depth, a cathodic activation function having a maximum depolarizing amplitude, and the anodal currents define, in combination, for the depth, an anodal activation function having a maximum hyperpolarizing amplitude greater than the maximum depolarizing amplitude. For some applications, for a 1 micron diameter myelinated axon within the nerve, the maximum hyperpolarizing amplitude is greater than or equal to 110% of the maximum depolarizing amplitude.

There is still further provided, in accordance with an embodiment of the present invention, apparatus for applying current to a nerve, including:

a housing, adapted to be placed in a vicinity of the nerve;

one or more cathodes, fixed to the housing, and configured to apply respective cathodic currents to the nerve; and

one or more anodes, fixed to the housing, and configured to apply respective anodal currents to the nerve,

wherein, for any given depth within the nerve, for a myelinated axon within the nerve of diameter less than 10 microns, the cathodic currents define, in combination, for the depth, a cathodic activation function having a maximum depolarizing amplitude, and the anodal currents define, in combination, for the depth, an anodal activation function having a maximum hyperpolarizing amplitude greater than the maximum depolarizing amplitude.

For some applications, for a 1 micron diameter myelinated axon within the nerve, the maximum hyperpolarizing amplitude is greater than or equal to 110% of the maximum depolarizing amplitude.

In an embodiment, the one or more cathodes include two or more cathodes, fixed to the housing such that no anodes are positioned longitudinally between the two or more cathodes.

In an embodiment, the cathodes and anodes are fixed to the housing such that no cathodes are positioned longitudinally between the one or more anodes.

In an embodiment, the one or more cathodes are fixed to the housing at respective cathodic locations; the one or more anodes include a single anode, fixed to the housing at an anodal location; the anode is configured to produce a virtual cathode effect at a virtual cathodic longitudinal site of the nerve, which is incapable of generating action potentials in the nerve; and the anodal location is between (a) the cathodic locations and (b) the virtual cathodic site. Alternatively, the anode is configured to produce the virtual cathode effect at the virtual cathodic longitudinal site which does not generate action potentials in more than 10% of axons of the nerve.

There is yet further provided, in accordance with an embodiment of the present invention, apparatus for applying current to a nerve, including:

a housing, adapted to be placed in a vicinity of the nerve;

one or more electrodes, fixed to the housing; and

two elongated end insulating elements, fixed to the housing such that all of the electrodes are longitudinally between the insulating elements, and adapted to be disposed with respect to the nerve such that each of the end insulating elements has a length in a direction parallel with the nerve of at least 2 mm.

For some applications, each of the end insulating elements has a maximum thickness along at least 75% of its length of less than 0.5 mm. For some applications, the end insulating elements are adapted to be positioned closer to the nerve than are the electrodes. For some applications, the housing includes one or more internal insulating elements that separate one or more of the electrodes, the internal insulating elements being adapted to be positioned closer to the nerve than the electrodes.

For some applications, the length of each of the end insulating elements is at least 3 mm, or at least 4 mm.

There is also provided, in accordance with an embodiment of the present invention, a method for applying current to a nerve, including:

applying at least one cathodic current and at least one anodal current to the nerve; and

passively electrically coupling at least two longitudinal sites of the nerve to one another.

There is further provided, in accordance with an embodiment of the present invention, a method for applying current to a nerve of a subject, including:

applying at least one cathodic current and at least one anodal current to the nerve; and

passively electrically coupling at least one site of the nerve to a remote location in a body of the subject at a distance of at least 1 cm from the nerve, such as at least 2 cm, e.g., at least 3 cm.

For some applications, passively electrically coupling includes electrically coupling a first end of a conducting element to the at least one site of the nerve, and inserting a second end of the conducting element into muscle tissue of the subject

There is still further provided, in accordance with an embodiment of the present invention, a method for applying current to a nerve having a radius, including:

applying two or more cathodic currents to the nerve at respective cathodic longitudinal sites; and

applying one or more anodal currents to the nerve at respective anodal longitudinal sites,

without applying anodal current to the nerve at any site longitudinally between the two or more cathodic longitudinal sites.

There is yet further provided, in accordance with an embodiment of the present invention, method for applying current to a nerve, including:

applying one or more cathodic currents to the nerve; and

applying one or more anodal currents to the nerve,

wherein, for any given depth within the nerve, for a myelinated axon within the nerve of diameter less than 10 microns, the cathodic currents define, in combination, for the depth, a cathodic activation function having a maximum depolarizing amplitude, and the anodal currents define, in combination, for the depth, an anodal activation function having a maximum hyperpolarizing amplitude greater than the maximum depolarizing amplitude.

There is also provided, in accordance with an embodiment of the present invention, a method for applying current to a nerve, including:

applying one or more currents to the nerve at respective longitudinal current-application sites of the nerve; and

applying electrical insulation to the nerve at two longitudinal insulation sites of the nerve, wherein all of the current-application sites are longitudinally between the two insulation sites, and each of the insulation sites has a length in a direction parallel with the nerve of at least 2 mm.

There is additionally provided, in accordance with an embodiment of the present invention, a method, including:

applying, to a stimulation site of a nerve, a spatially-periodic stimulating field, configured to induce, in small fibers of the nerve, action potentials that propagate from the stimulation site towards a target site and away from the target site; and

applying, to an inhibition site of the nerve, a spatially-periodic non-stimulating field, configured to partially depolarize at the inhibition site the small fibers of the nerve, without initiating action potentials therein,

the partial depolarization of the small fibers being sufficient to inhibit the action potentials propagating away from the target site from continuing to propagate beyond the inhibition site,

the stimulation site being between the target site and the inhibition site.

In some applications of the present invention, an electrode assembly comprises a cuff and one or more electrodes. The cuff comprises an electrically-insulating material, and is shaped so as to define a tubular housing. The housing is shaped so as to define two edges and a longitudinal slit between the two edges. The slit and edges extend along an entire length of the cuff. The housing is configured to assume (a) an open position, in which the two edges do not touch each other, and (b) a closed position, in which (i) respective contact surfaces of the two edges touch each other, and (ii) the housing defines an inner surface that faces and surrounds a longitudinal axis of the housing. The electrodes are fixed to the inner surface.

For some applications, the cuff is shaped so as to define three or more annular insulating elements that extend toward the axis from the inner surface of the housing at respective longitudinal positions along housing. When the housing is in the closed position, the inner surface and pairs of the insulating elements are shaped so as to define, at respective longitudinal positions along the housing, respective chambers open toward the axis. For some applications, one or more of the electrodes are fixed within respective ones of chambers.

For some applications, the contact surfaces of the two edges extend toward the axis and protrude into the chambers. This configuration provides greater surface contact between the contact surfaces than if the contact surfaces did not extend into the chambers. This greater surface contact causes the contact surfaces to form a better electrical seal with each other, thereby reducing current leakage from the cuff.

For some applications, at least one of the electrodes comprises a strip of metal foil having two end portions and a central portion between the end portions. The central portion is disposed against the inner surface of the housing such that, when the housing is in the closed position, the central portion forms a partial ring around the axis that defines an exposed, electrically-conductive surface of the central portion, which exposed surface faces the axis. At least one of the end portions is shaped so as to define a curved portion that is embedded in and completely surrounded by the insulating material of the cuff, thereby fixing the end portion to the insulating material. This curved portion helps firmly secure the electrode to the insulating material of the cuff. Typically, the curved portion has an average radius of curvature that is less than 10% of a length of the central portion measured around axis, such as less than 5%, e.g., less than 3%.

There is therefore provided, in accordance with an application of the present invention, apparatus including an electrode assembly, which includes:

one or more electrodes; and

a cuff, which includes an electrically insulating material, and which is shaped so as to define:
a tubular housing that defines a longitudinal axis therealong, the housing shaped so as to define two edges and a slit between the two edges, which slit and edges extend along an entire length of the cuff, wherein the housing is configured to assume (a) an open position, in which the two edges do not touch each other, and (b) a closed position, in which (i) respective contact surfaces of the two edges touch each other, and (ii) the housing defines an inner surface that faces and surrounds the axis, to which inner surface the electrodes are fixed, and
three or more annular insulating elements that extend toward the axis from the inner surface of the housing at respective longitudinal positions along the housing, such that, when the housing is in the closed position, the inner surface of the housing and pairs of the insulating elements are shaped so as to define, at respective longitudinal positions along the housing, respective chambers open toward the axis,

wherein the housing is shaped such that the contact surfaces of the two edges extend toward the axis and protrude into the chambers.

For some applications, the housing is shaped such that the slit and the edges extend parallel to the axis along the entire length of the cuff.

For some applications, the cuff is shaped such that the insulating elements and the contact surfaces extend to a same average distance from the axis.

For some applications, the cuff is shaped such that a first average distance that the contact surfaces extend from the inner surface toward the axis is less than a second average distance that the insulating elements extend from the inner surface toward the axis.

For some applications, the housing is shaped such that an average distance that the first contact surfaces extend from the inner surface toward the axis is at least 0.6 mm.

For any of the applications described above, the electrodes may be shaped as partial rings, both when the housing is in the open position and when the housing is in the closed position.

For any of the applications described above, two of the insulating elements may be disposed at respective longitudinal ends of the tubular housing.

For any of the applications described above, the entire length of the cuff may be between 1 and 40 mm, such as between 5 and 20 mm.

For any of the applications described above, at least one of the electrodes may be fixed to the inner surface within one of the chambers.

For any of the applications described above, the cuff may be shaped so as to be placeable around an elliptical cylinder having a major axis that is between 1 and 8 mm and a minor axis that is between 0.5 and 6 mm, and to assume the closed position when thus placed.

There is further provided, in accordance with an application of the present invention, apparatus including an electrode assembly, which includes:

a cuff, which includes an electrically insulating material, and which is shaped so as to define a tubular housing that defines a longitudinal axis therealong, the housing shaped so as to define two edges and a slit between the two edges, which slit and edges extend along an entire length of the cuff, wherein the housing is configured to assume (a) an open position, in which the two edges do not touch each other, and (b) a closed position, in which (i) respective contact surfaces of the two edges touch each other, and (ii) the housing defines an inner surface that faces and surrounds the axis; and

one or more electrodes, at least one of which includes a strip of metal foil having two end portions and a central portion between the end portions,

wherein the central portion is disposed against the inner surface of the housing such that, when the housing is in the closed position, the central portion forms a partial ring around the axis that defines an exposed surface of the central portion, which exposed surface faces the axis, and

wherein at least one of the end portions is shaped so as to define a curved portion that is embedded in and completely surrounded by the insulating material, thereby fixing the end portion to the insulating material, the curved portion having an average radius of curvature that is less than 10% of a length of the central portion measured around the axis.

For some applications, the average radius of curvature of the curved portion is less than 5% of the length of the central portion measured around the axis.

For some applications, the housing is shaped such that the slit and the edges extend parallel to the axis along the entire length of the cuff.

For any of the applications described above, the average radius of curvature of the curved portion may be less than 1.5 mm, such as less than 0.5 mm.

For any of the applications described above, the strip of metal foil may be shaped such that a direction of curvature of the curved portion is opposite a general direction of curvature of the central portion.

For any of the applications described above, the curved portion may be shaped so as to define an arc that subtends an angle of at least 90 degrees, such as at least 180 degrees, e.g., at least 270 degrees, such as at least 360 degrees.

For any of the applications described above, the curved portion, if straightened, may have a length of at least 1.5 mm, measured in a direction perpendicular to the axis. Alternatively or additionally, for any of the applications described above, the curved portion, if straightened, may have a length equal to at least 5% of an entire length of the strip of metal, if straightened, which lengths are measured in a direction perpendicular to the axis.

For any of the applications described above, the at least one of the two end portions may be a first one of the two end portions, the curved portion may be a first curved portion, and a second one of the two end portions may be shaped so as to define a second curved portion that is embedded in and completely surrounded by the insulating material, thereby fixing the second end portion to the insulating material, the second curved portion having an average radius of curvature that is less than 10% of the length of the central portion measured around the axis.

There is still further provided, in accordance with an application of the present invention, a method including:

providing an electrode assembly that includes (1) one or more electrodes, and (2) a cuff, which includes an electrically insulating material, and which is shaped so as to define (A) a tubular housing that defines a longitudinal axis therealong, the housing shaped so as to define two edges and a slit between the two edges, which slit and edges extend an entire length of the cuff, wherein the housing is configured to assume (a) an open position, in which the two edges do not touch each other, and (b) a closed position, in which (i) respective contact surfaces of the two edges touch each other, and (ii) the housing defines an inner surface that faces and surrounds the axis, to which inner surface the electrodes are fixed, and (B) three or more annular insulating elements that extend toward the axis from the inner surface of the housing at respective longitudinal positions along the housing, such that, when the housing is in the closed position, the inner surface of the housing and pairs of the insulating elements are shaped so as to define, at respective longitudinal positions along the housing, respective chambers open toward the axis, wherein the housing is shaped such that the contact surfaces of the two edges extend toward the axis and protrude into the chambers;

while the housing is in the open position, placing the cuff around tubular body tissue of a subject; and

coupling the cuff to the tubular body tissue by causing the housing to assume the closed position.

There is additionally provided, in accordance with an application of the present invention, a method including:

providing an electrode assembly that includes (1) a cuff, which includes an electrically insulating material, and which is shaped so as to define a tubular housing that defines a longitudinal axis therealong, the housing shaped so as to define two edges and a slit between the two edges, which slit and edges extend an entire length of the cuff, wherein the housing is configured to assume (a) an open position, in which the two edges do not touch each other, and (b) a closed position, in which (i) respective contact surfaces of the two edges touch each other, and (ii) the housing defines an inner surface that faces and surrounds the axis, and (2) one or more electrodes, at least one of which includes a strip of metal foil having two end portions and a central portion between the end portions, wherein the central portion is disposed against the inner surface of the housing such that, when the housing is in the closed position, the central portion forms a partial ring around the axis that defines an exposed surface of the central portion, which exposed surface faces the axis, and wherein at least one of the end portions is shaped so as to define a curved portion that is embedded in and completely surrounded by the insulating material, thereby fixing the end portion to the insulating material, the curved portion having an average radius of curvature that is less than 10% of the length of the central portion measured around the axis;

while the housing is in the open position, placing the cuff around tubular body tissue of a subject; and

coupling the cuff to the tubular body tissue by causing the housing to assume the closed position.

The present invention will be more fully understood from the following detailed description of the preferred embodiments thereof, taken together with the drawings, a brief description of which follows.

### BRIEF DESCRIPTION OF THE DRAWINGS

FIG. 1 is a schematic diagram illustrating a stimulation device, according to a preferred embodiment of the present invention;

FIGS. 2A and 2B are electronic diagrams of a circuit comprised in the device of FIG. 1, according to preferred embodiments of the present invention;

FIG. 3 shows graphs of voltage versus time for different types of pulses generated in the circuit, according to a preferred embodiment of the present invention;

FIG. 4 shows graphs of voltage versus time for elements of the circuit when one of the types of pulses illustrated in FIG. 3 is generated, according to a preferred embodiment of the present invention;

FIG. 5 is a flowchart showing a process for charging a capacitor in the circuit, according to a preferred embodiment of the present invention;

FIG. 6 is a schematic diagram illustrating an alternate stimulation device, according to a preferred embodiment of the present invention;

FIG. 7 is a schematic electronic diagram of a calibration circuit, according to a preferred embodiment of the present invention;

FIGS. 8A and 8B are electronic diagrams of an alternative circuit comprised in the device of FIG. 1, according to preferred embodiments of the present invention;

FIGS. 9 and 10 are voltage vs. time graphs for the circuit of FIGS. 8A and 8B;

FIG. 11 is a flowchart showing steps involved in setting a voltage in the circuit of FIGS. 8A and 8B, according to a preferred embodiment of the present invention;

FIG. 12 are voltage vs. time graphs illustrating the operation of the flowchart of FIG. 11;

FIGS. 13A-15 are schematic, cross-sectional illustration of electrode assemblies for applying current to a nerve, in accordance with respective embodiments of the present invention;

FIGS. 16 and 17 are graphs modeling calculated activation functions when current is applied using an electrode assembly similar to that of FIG. 15, in accordance with an embodiment of the present invention;

FIG. 18 is a schematic, cross-sectional illustration of another electrode assembly for applying current to a nerve, in accordance with an embodiment of the present invention;

FIG. 19 is a schematic, cross-sectional illustration of an electrode assembly for applying Electrode Array Selective Stimulation (EASS) to a nerve, in accordance with an embodiment of the present invention;

FIG. 20 is a schematic illustration of a selective stimulation EASS system, in accordance with an embodiment of the present invention;

FIGS. 21A-C are schematic illustrations of an electrode assembly, in accordance with an application of the present invention;

FIGS. 22A and 22B are schematic illustrations of a cuff of the electrode assembly of FIGS. 21A-C in slightly open and closed positions, respectively, in accordance with an application of the present invention; and

FIG. 23 is a schematic cut-away illustration of the cuff of FIGS. 22A and 22B, in accordance with an application of the present invention.

### DETAILED DESCRIPTION OF APPLICATION

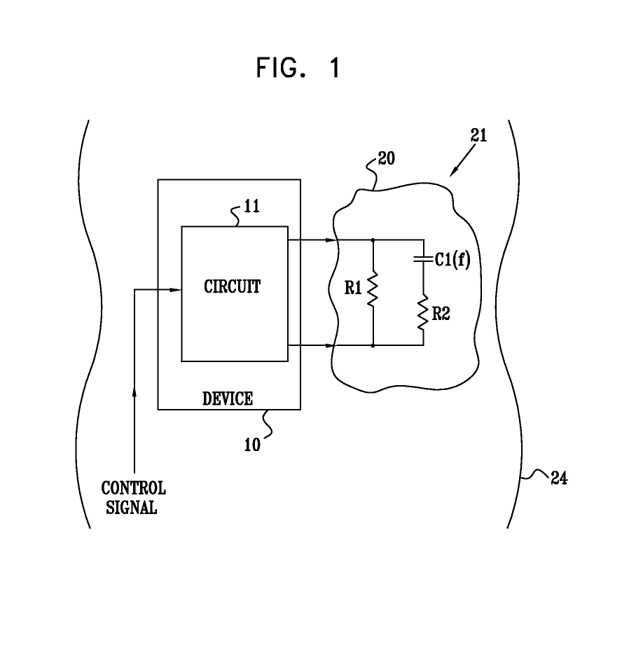

Reference is now made to FIG. 1, which is a schematic diagram illustrating a stimulation device 10, according to a preferred embodiment of the present invention. Device 10 comprises a circuit 11 which is used to generate electrical waveforms, the waveforms in turn stimulating tissue 20 of a subject 24. Preferably, tissue 20 comprises a muscle, most preferably a sphincter muscle, or a nerve of the subject, although it will be appreciated that tissue 20 may comprise any tissue of subject 24. Device 10 is preferably implanted in the subject, and after implantation and adjustment, is most preferably operated by a control signal generated by subject 24 and input to circuit 11. Circuit 11 may be implemented as discrete components, or as a custom-built component such as an application specific integrated circuit (ASIC), or as a combination of discrete and custom-built components. By way of example, the description hereinbelow applies when circuit 11 comprises discrete components.

An equivalent circuit 21 for tissue 20 comprises an approximately ohmic resistor R2 in series with an interface capacitor having a capacitance C1(f), where f is a frequency of a stimulation applied to the tissue. An approximately ohmic resistor R1 is coupled in parallel with R2 and C1(f). Typical values for R1, R2, and C1(f) are of the order of 1 MΩ, 200Ω, and 40 μF. It will be understood that the values for R1, R2, and C1(f) are dependent on factors such as the type, state, and size of tissue 20. Furthermore, the values of R1, R2, and C1(f) are also typically highly dependent on the shape, size, and type of material of the electrodes used to apply stimulation to tissue 20, as well as on the frequency and voltage of the stimulation applied to the interface capacitor.

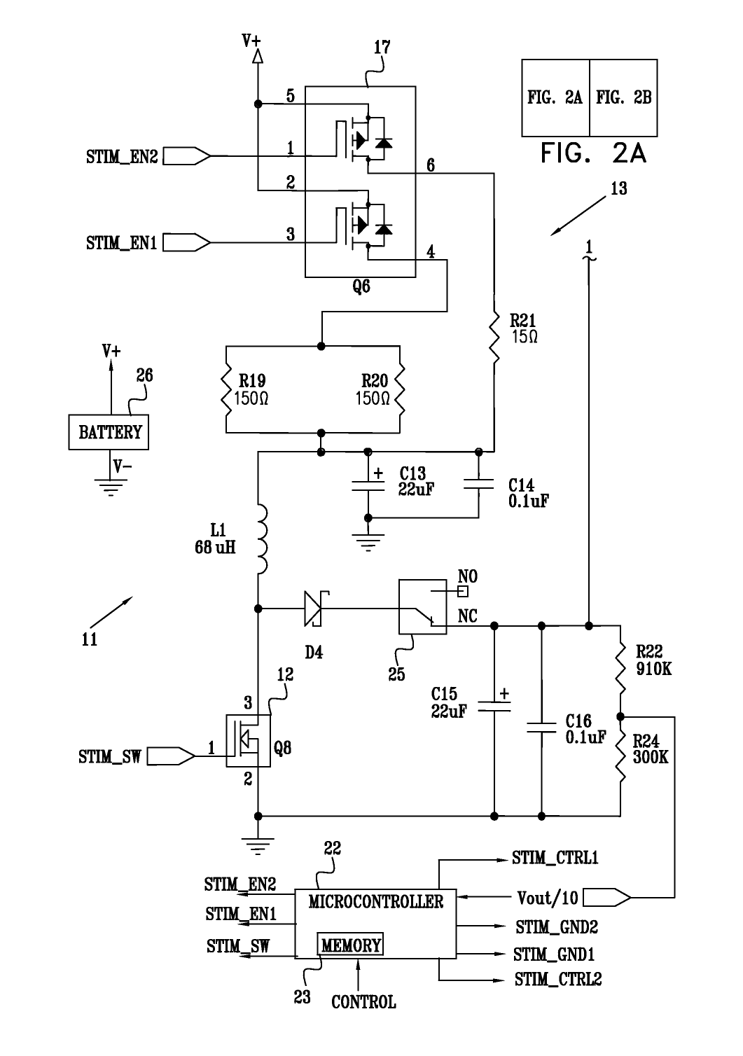

FIGS. 2A and 2B are electronic diagrams of circuit 11, according to preferred embodiments of the present invention. Circuit 11 is powered by a battery 26, which preferably has a maximum voltage approximately equal to 3.2 V. Circuit 11 comprises a charging circuitry section 13 (FIG. 2A) and a switching circuitry section 15 (FIG. 2B). A micro-controller (MC) 22 acts as an overall controller of circuit 11, using as an input the control signal referred to in FIG. 1. Preferably, MC 22 comprises an XE88LC01 produced by Xemics SA of Neuchatel, Switzerland, although any other suitable micro-controller may be used. MC 22 includes, inter alia, a memory 23 wherein parameters for operating circuit 11 may be stored, and an analog-to-digital converter (ADC) 25, which converts analog voltage levels to digital values.

Charging circuitry section 13 operates in two modes, a direct current (DC) mode and an alternating current (AC) mode. The DC mode is activated when a switching device 17 (Q6) is connected so that a positive terminal of battery 26 is coupled to parallel resistors R19 and R20, each resistor most preferably having approximate values of 150Ω. In the DC mode the positive terminal is de-coupled from resistor R21, which most preferably has an approximate value of 15Ω. Values of resistors R19 and R20 are chosen so as to prevent a current from battery 26 exceeding an allowed maximum battery current value. Switch 17 is most preferably implemented from an FDC6306P produced by Fairchild Semiconductor Corporation of South Portland, Me. and the DC mode is activated by micro-controller 22 enabling control signal STIM_EN1 and disabling STIM_EN2.

In the DC mode, a switch 12 (Q8)—most preferably implemented from a field effect transistor (FET) such as an IRLML2402 produced by International Rectifier Corporation of El Segundo, Calif.—is open, so that battery 26 charges capacitors C13 and C15, which both preferably have a value approximately equal to 22 μF, in an RLC circuit formed by resistors R19, R20, an inductor L1, and the capacitors. During operation of circuit 11, the DC mode most preferably operates for a time period, defined by MC 22, no greater than approximately 3·τ seconds, where τ is a charging time constant equal to the RC value of R19, R20, C13, and C15, i.e., 75·44·10⁻⁶. It will be understood that by charging the capacitors for this length of time (when the capacitors start in a completely discharged state), C15 achieves approximately 95% of the potential of battery 26.

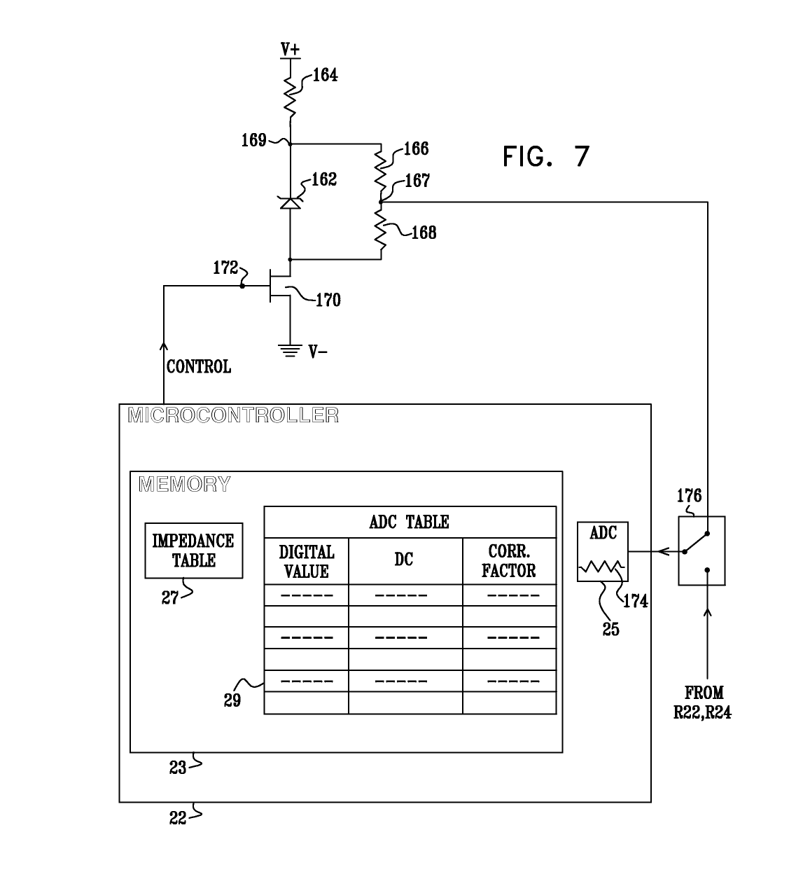

The AC mode is activated when switch 17 couples the positive terminal of battery 26 to R21, while R19 and R20 remain coupled as for the DC mode. MC 22 activates the AC mode by enabling control signals STIM_EN1 and STIM_EN2. In the AC mode switch 12 is rapidly switched closed and open by a rectangular signal STIM_SW, generated by the micro-controller, alternating between on and off states. Most preferably, STIM_SW is on for approximately 4 μs, and off for approximately the same time interval. An alternative timing for STIM_SW is described with reference to FIGS. 8A and 8B below. When switch 12 is closed, inductor L1, preferably having an approximate value of 68 μH, is energized by current through the inductor flowing to ground. When switch 12 is open, current from inductor L1 is diverted via a diode D4 to charge capacitor C15. Assuming that a combined resistance of an internal resistance of inductor L1 and switch 12 is approximately 600 mΩ, approximately 99.7% of the current flowing in the inductor charges capacitor C15. Preferably, a switch 19, most preferably a Reed switch, is positioned before capacitor C15 for use as a safety cut-out.

It will be appreciated that operating circuit 11 in the AC mode as described above enables capacitor C15 to be charged to a high voltage, most preferably of the order of 9 V, which is substantially independent of a voltage supplied from battery 26 and which is only limited by the time during which the AC mode is operative. It will also be appreciated that from an energy efficiency point of view, it is preferable to use the AC mode rather than the DC mode for charging capacitor C15. The charging rates for both modes may be calculated from values of elements of circuit 11, including a potential delivered by battery 26, as will be apparent to those skilled in the art. The rates are preferably stored in memory 23. In some preferred embodiments of the present invention, which mode is used, and for how long the mode is implemented, is a function of a voltage differential to which capacitor C15 is to be charged, respective rates of charging for the DC and AC modes, and a time during which the capacitor is available for charging. A more detailed description of capacitor C15 charging by utilizing stored charging rates is given below.

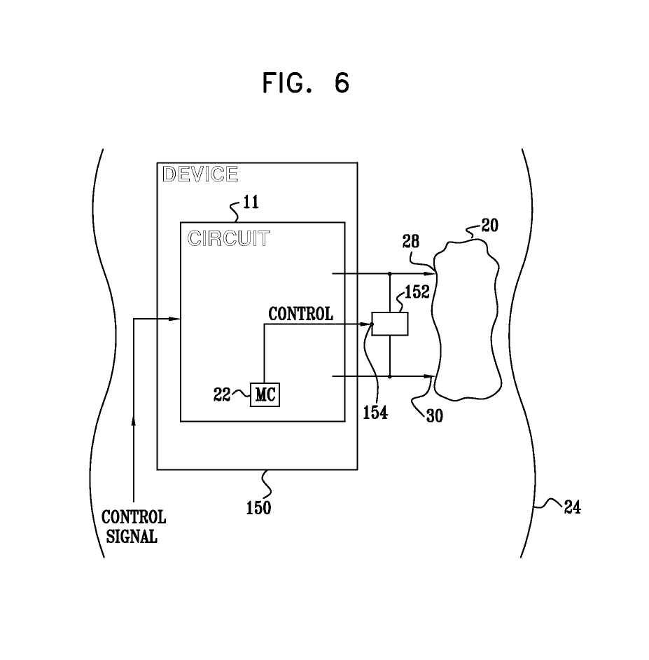

During operation of circuit 11, micro-controller 22 monitors the voltage across capacitor C15 using resistors R22 and R24 coupled in series across C15, the resistors acting as a voltage divider. The monitored voltage is converted to digital values using ADC 25. Preferably, memory 23 also comprises an ADC look-up table 29, the function and composition of which is described with reference to FIG. 7 below.

In section 15 (FIG. 2B), capacitor C15 is used to generate pulses at a first electrode 28 and a second electrode 30 implanted in tissue 20, via operation of switches 14, 16, and 18. Capacitor C15 thus acts as a stimulation source for the implanted electrodes, and is herein also referred to as a stimulation capacitor. Switches 14 and 18 are implemented as single pole single throw (SPST) switches, preferably integrated load switches FDC6324L produced by Fairchild Semiconductor Corporation, although any other suitable switches may be used. Switch 16 comprises two separate SPST switches 34 and 36, preferably implemented from an integrated load switch FDC6324L, produced by Fairchild Semiconductor Corporation.

Table I below shows states of switches 14, 18, 34, and 36, and respective control signals, as used to generate a positive-going pulse, where electrode 28 is positive with respect to electrode 30, and a negative-going pulse, where electrode 28 is negative with respect to electrode 30.

TABLE I

| | | Switch state | |
|---|---|---|---|
| | | Positive-going | Negative-going |
| Switch | Switch Control | pulse | pulse |
| Switch 14 | STIM_CTRL1+ | closed | open |
| Switch 18 | STIM_CTRL2− | open | closed |
| Switch 36 | STIM_GND1+ | closed | open |
| Switch 34 | STIM_GND2− | open | closed |

Resistors R29 and R33, each approximately equal to 15Ω, respectively act as current limiting resistors for positive-going and negative-going pulses. Current limitation may typically be required in the event of an inadvertent short between electrode 28 and electrode 30.

MC 22 sets switches 14, 18, 36, and 34 according to Table I, in order to produce pulses as required. MC 22 is also able to monitor an impedance of tissue 20, by measuring the discharge of stimulation capacitor C15 as pulses are generated in tissue 20, as described in more detail below.

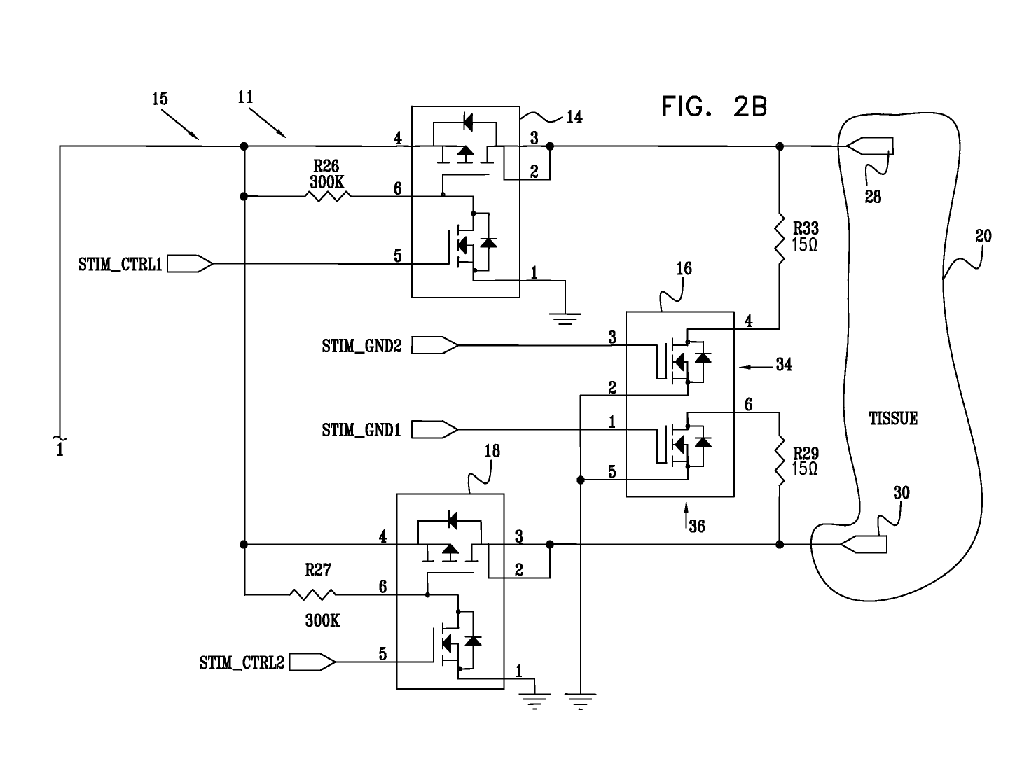

FIG. 3 shows graphs of voltage versus time for different types of pulses generated in circuit 11, according to a preferred embodiment of the present invention. A graph 50 illustrates biphasic pulses 55 generated at electrodes 28 and 30, the biphasic pulses having a period of τ₁. Each biphasic pulse 55 is formed from a uni-directional pulse 54 and a uni-directional pulse 56 which are substantially mirror images of each other, having substantially equal pulse width times τ₂. Pulse 54 is a positive-going pulse having an initial potential Vₛₜᵢₘ, pulse 56 is a negative-going pulse having an initial potential −Vₛₜᵢₘ. A time τ₃ between pulses 54 and 56 is very much less than the period τ₁ of the pulses.

A graph 60 illustrates alternating pulses 64 and 66 generated at electrodes 28 and 30, having a period of τ₄. Pulses 64 and 66 are substantially mirror images of each other, having substantially equal pulse width times τ₅. Pulse 64 is a uni-directional positive-going pulse having an initial potential Vₛₜᵢₘ, pulse 66 is a uni-directional negative-going pulse having an initial potential −Vₛₜᵢₘ. However, unlike pulses 54 and 56, a time τ₆ between alternating pulses 64 and 66 is approximately equal to half the period τ₄ of the pulses.

Most preferably, initial values for Vₛₜᵢₘ and times τ₁, τ₂, τ₃, τ₄, τ₅, and τ₆ are implemented by an operator of device 10, in conjunction with feedback from subject 24, in order to correctly stimulate tissue 20, and the values are stored in memory 23. It will be appreciated that more than one set of initial values of Vₛₜᵢₘ and times τ₁, τ₂, τ₃, τ₄, τ₅, and τ₆ may be stored, and each particular set of values may be implemented by subject 24 or by the operator. For example, if tissue 20 comprises a sphincter muscle of the urinary tract, a first set of values may have Vₛₜᵢₘ equal to approximately 6V, and times τ₁, τ₂, τ₃, τ₄, τ₅, and τ₆ equal to approximately 25 ms, 1 μs, 500 μs, 50 ms, 1 μs, and 25 ms respectively. A second set of values may have Vₛₜᵢₘ equal to approximately 2V, and times τ₁, τ₂, τ₃, τ₄, τ₅, and τ₆ equal to approximately 100 ms, 1 μs, 500 μs, 200 ms, 1 μs, and 100 ms respectively. Micro-controller 22 may use either or both sets, and/or other similar sets of parameters, in order to stimulate tissue 20.

Micro-controller 22 implements both alternating and biphasic pulses by operating switches 17 and/or 12 to charge stimulation capacitor C15 to a voltage Vₛₜᵢₘ. Once the capacitor has been charged, switches 14, 16, and 18 are operated, as described with reference to Table I above, to generate the pulses.

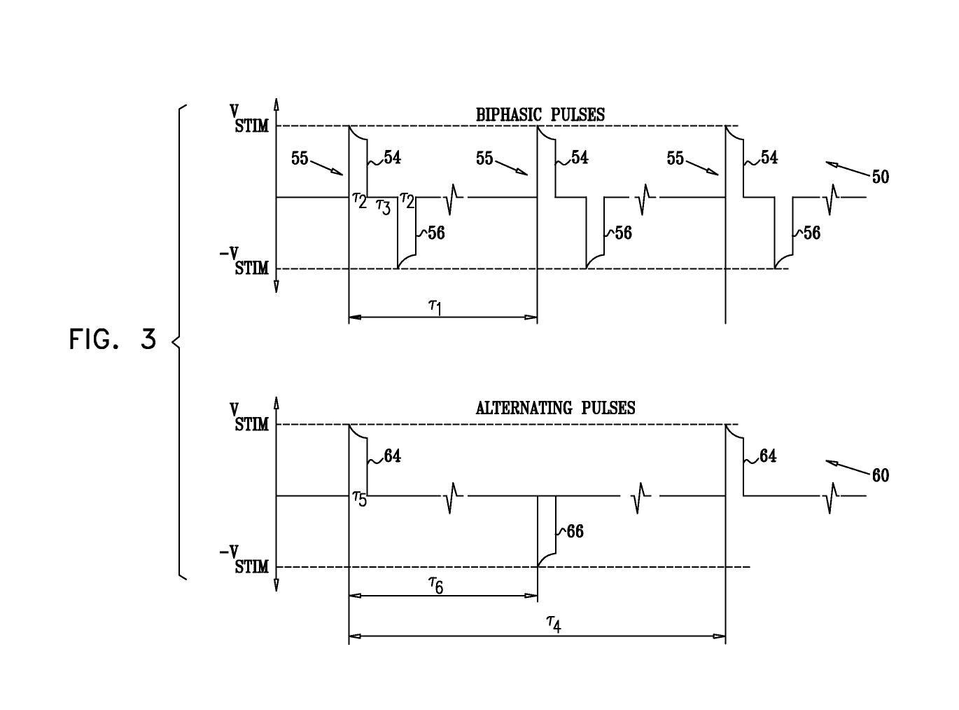

FIG. 4 shows graphs of voltage versus time for elements of circuit 11 when the biphasic pulses illustrated in FIG. 3 are generated, according to a preferred embodiment of the present invention. Generally similar graphs of voltage versus time for the elements apply for generation of alternating pulses. Typically, circuit 11 generates a sequence of pulses (biphasic or alternating) on receipt of the control signal (FIG. 2A) at MC 22. Preferably, if tissue 20 comprises a sphincter muscle, the control signal is derived from a pressure sensor activated by subject 24. Most preferably, a level of the control signal is used to determine which type of pulses (biphasic and/or alternating) are produced by circuit 11.

The graphs of FIG. 4 illustrate voltages versus time for capacitor C15 in a period 80 before the capacitor reaches a quasi-steady state, and in a period 82 when the capacitor is in the quasi-steady state. By way of example, τ₁, τ₂, and τ₃ for the biphasic pulses of graph 50 are assumed to be approximately 20 ms, 1 μs, and 500 μs respectively, battery 26 is assumed to supply 3V, and the biphasic pulses have a quasi-steady state amplitude of 3.5V, although it will be appreciated that circuit 11 may implement other values for these parameters.

A graph 70 represents the voltage on capacitor C15. At the beginning of a period 84, at which time circuit 11 is required to generate a biphasic pulse, capacitor C15 has been charged to 3V. The biphasic pulse generated in period 84 causes capacitor C15 to discharge at the end of period 84 to 2V.

At the end of period 84, MC 22 preferably evaluates periods during which the DC mode and the AC mode are to be implemented. Preferably, the period for the AC mode is made as large as possible, since charging using the AC mode is more efficient than using the DC mode. The evaluation is based on the charging rates for the DC mode the AC mode (stored in memory 23), a desired voltage differential to be achieved, and an available time for charging. Thus, for a time period 86 following period 84, the desired voltage differential is 1.5V and the available time is approximately 20 ms. By way of example, MC 22 is assumed to set a DC mode period 88 of approximately 8 ms, and an AC mode period 90 of approximately 12 ms.

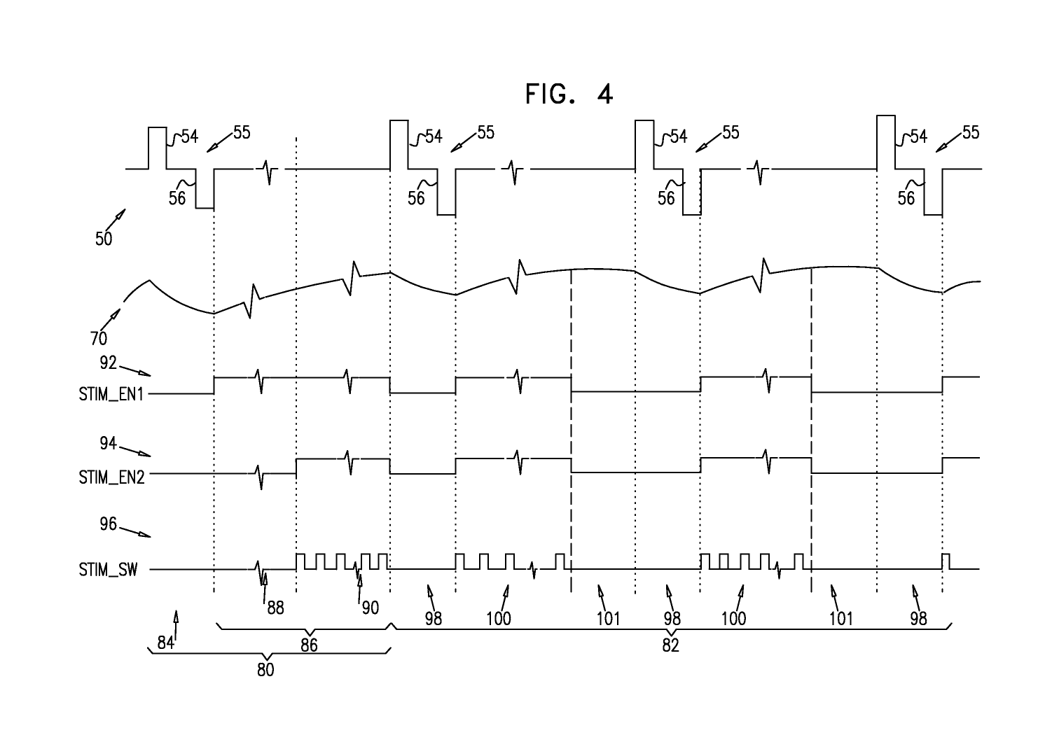

An alternative method by which MC 22 is able to charge stimulation capacitor C15 is described with reference to FIG. 5 below.

Voltage waveforms for STIM_EN1, STIM_EN2, and STIM_SW are shown in graphs 92, 94, and 96 respectively. Time period 88 is implemented by enabling STIM_EN1. Time period 90 is implemented by enabling STIM_EN1 and STIM_EN2. During period 90, STIM_SW pulses activate switch 12, as described above, and at the end of period 90 capacitor C15 has charged to 3.5V, so that quasi-steady state period 82 begins.

During periods 98, circuit 11 generates biphasic pulses of amplitude 3.5V, and capacitor C15 discharges from 3.5V to 2.5V. At the end of each period 98, MC 22 preferably makes an evaluation, substantially similar to that described above for the end of period 84, for charging periods 100. Herein MC 22 is assumed to set an AC mode period of approximately 20 ms, so that the DC mode is not implemented for periods 100, as is illustrated by graphs 92, 94, and 96. If capacitor C15 charges to the required voltage, STIM_EN1 and STIM_EN2 are disabled, and STIM_SW terminates, as shown in periods 101. Alternatively, MC 22 implements the method of FIG. 5 for charging capacitor C15.

It will be appreciated that by being able to use either or both AC and DC charging modes, circuit 11 is able to reach a quasi-steady state quickly and efficiently, and is also able to maintain the quasi-steady state with a minimal waste of energy. Both factors are important for optimal implementation of battery powered implanted devices delivering sequences of pulses.

FIG. 5 is a flowchart showing an alternative process 99 for charging capacitor C15, before generating pulses 55, 64, and 66 (FIG. 3), according to a preferred embodiment of the present invention. Process 99 does not rely on knowledge of charging rates of the DC and AC mode of circuit 11.

In an initial step 102 of the process, the voltage Vₒᵤₜ across C15 is measured by MC 22 sampling the potential at the junction of R22 and R24.

In a first decision step 103, MC 22 compares the value of Vₒᵤₜ with a required voltage stimulation value Vₛₜᵢₘ stored as described above in memory 23. If Vₒᵤₜ is greater than or equal to Vₛₜᵢₘ, process 99 ends. If Vₒᵤₜ is less than Vₛₜᵢₘ, process 99 continues to a second decision step 104.

In decision step 104, MC 22 compares the value of Vₒᵤₜ determined in step 102 with a limit value which is set to be a function of a voltage delivered by battery 26. Most preferably, the limit value is implemented to be approximately 90% of the battery voltage, although another value, less than the battery voltage, may be used. If Vₒᵤₜ is greater than or equal to the limit value, process 99 continues to a third decision step 108. If Vₒᵤₜ is less than the limit value, in a DC charging step 106 the DC charging phase of section 13 is implemented, by setting STIM_EN_{—}1, and ensuring that STIM_EN_{—}2 is not set. DC charging step 106 is preferably activated for a period of approximately 1 ms, after which step 102 is implemented. The process of cycling through steps 102, 103, 104, and 106 continues for up to five times, or until step 104 is not satisfied.

In third decision step 108, MC 22 compares the value of Vₒᵤₜ with Vₛₜᵢₘ. If Vₒᵤₜ<Vₛₜᵢₘ, then MC 22 initiates an AC charging step 110, during which the AC charging phase of section 13 is implemented, by setting STIM_EN_{—1 and STIM}_EN_2.

During step 110, switch Q8 is toggled between on and off states, as described above. When Q8 is in its off state, MC 22 measures Vₒᵤₜ, as described for step 102. The process of AC charging continues until step 108 is no longer valid, i.e., Vₒᵤₜ=Vₛₜᵢₘ. Alternatively, the AC charging continues until the time at which a pulse is to be generated, corresponding to the end of periods τ₁ or τ₆ (FIG. 3), at which point process 99 terminates.

It will be appreciated that, depending on the differential potential to which capacitor C15 is to be charged, and on the time period available for charging, process 99 may invoke operation of the AC or the DC circuits of section 13, or both circuits.

Returning to FIGS. 2A, 2B, and 3, MC 22 measures a potential on stimulation capacitor C15 at the end of each pulse. MC 22 uses this value, the initial value Vₛₜᵢₘ of the pulse, and the period τ₂ for biphasic pulses (or τ₅ for alternating pulses), to evaluate an impedance of tissue 20, most preferably from an impedance look-up table 27 stored in memory 23. (MC 22 may obtain values of Vₛₜᵢₘ, τ₂ and τ₅ from memory 23.) During periods when MC 22 determines that the impedance of tissue 20 is substantially constant, values of Vₛₜᵢₘ, τ₂ and τ₅ are not altered In the event that the impedance does alter, MC 22 most preferably alters the value of Vₛₜᵢₘ so that an average current generated by the pulses is substantially constant. Alternatively or additionally, MC 22 alters the width of the pulses so that the average current is substantially constant. Any altered values are stored in memory 23, and are used by MC 22 in subsequent pulse generation. Measurements of the impedance, as described above, enable MC 22 to detect open or short circuits, such as may occur by misplacement of one of electrodes 28, 30.

It will be appreciated that both biphasic and alternating pulses generated by preferred embodiments of the present invention comprise sequential pulses which are substantially equal in magnitude but opposite in direction to each other. Thus, charge transfer when the pulses are applied to tissue 20 is substantially equal in magnitude but opposite in direction. Thus, substantially no electrolysis occurs during stimulation to the tissue, because interface capacitance C1(f) is discharged by the opposite sign pulses comprised in the biphasic or alternating pulses. This is in contrast to prior art systems applying mono-phase pulses to tissue 20, in which cases capacitance C1(f) slowly discharges through resistors R1 and R2, effectively causing electrolysis of tissue 20.

It will also be appreciated that by measuring the impedance of tissue 20 as described above, and by adjusting stimulation parameters responsive to the impedance, there is no need for measuring or estimating current in tissue 20 by a separate current-sensing circuit, as is implemented in systems known in the art for stimulating tissue.

The biphasic pulses described hereinabove, and illustrated in graph 50 (FIG. 3 and FIG. 4), each comprise a positive-going pulse followed by a negative-going pulse. Such a non-alternating sequence of biphasic pulses may be represented as (+−), (+−), (+−), (+−), . . . . It will be appreciated that the biphasic pulses may each comprise a negative-going pulse followed by a positive-going pulse, which non-alternating sequence may be represented as (−+), (−+), (−+), (−+), . . . . It will be further appreciated that the biphasic pulses may be alternated in a regular or an irregular sequence, so that alternating sequences of biphasic pulses of the form (+−), (−+), (+−), (−+), (+−), . . . , or (+−), (+−), (−+), (+−), (+−), . . . , or of any other regular or irregular sequence may be generated. The application of alternating sequences of biphasic pulses may reduce electrolysis effects at electrodes 28 and 30 (FIG. 2B). All forms and combinations of non-alternating and alternating sequences of biphasic pulses are assumed to be comprised within the scope of the present invention.

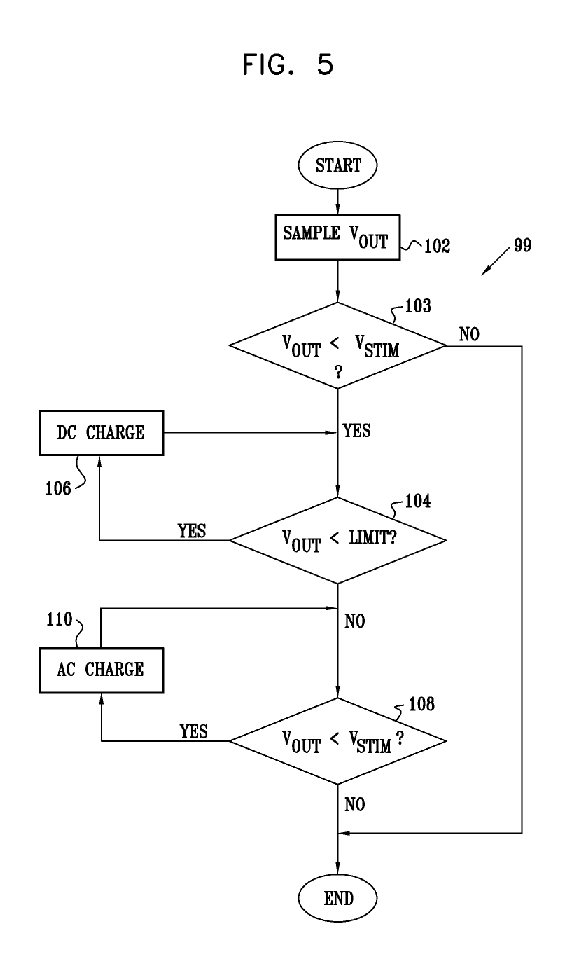

FIG. 6 is a schematic diagram illustrating an alternate stimulation device 150, according to a preferred embodiment of the present invention. Apart from the differences described below, the operation of device 150 is generally similar to that of device 10 (FIGS. 1, 2A, 2B, and 3), so that elements indicated by the same reference numerals in both devices 150 and 10 are generally identical in construction and in operation. Device 150 comprises a controllable element 152 which is connected between electrodes 28 and 30, and which is able to act as an effective short-circuit therebetween, responsive to a control signal from MC 22. Element 152 preferably comprises a resistor in series with an FET such as an IRLML2402, or alternatively any other set of electronic components, such as will be apparent to those skilled in the art, which are able to act as a controllable short-circuit. The resistance of element 152, when it is operating as a short-circuit, is preferably significantly less than R1, typically having a value of the order of hundreds of ohms. Most preferably, the resistance of element 152 is set by MC 22 so that no anodal break excitation occurs in tissue 20.

The control signal for element 152 is input to a control electrode 154 of the element, the control electrode comprising a gate of an FET if element 152 is implemented therefrom. Most preferably, MC 22 activates the control signal directly after a biphasic pulse or an alternating pulse has been impressed on electrodes 28 and 30. By activating the control signal directly after the pulses, and thus short-circuiting the electrodes, the level of stimulation to tissue 20 is more effectively controlled. Furthermore, any residual charge on the electrodes from the pulses charging interface capacitance C1(f) is substantially neutralized, so reducing electrolysis caused by discharge of the capacitance.

FIG. 7 is a schematic electronic diagram of a calibration circuit 160, according to a preferred embodiment of the present invention. Circuit 160 is most preferably included in device 10 and device 150, and is used as a reference for correcting inaccuracies in the operation of ADC 25. Apart from the differences described below when circuit 160 is implemented, operations of devices 10 and 150 are generally as described above with reference to FIGS. 1, 2A, 2B, 3 and 6.

Implanted devices such as device 10 and device 150 are expected to operate for a number of years before battery recharging, or before device or battery replacement. The device thus needs to operate over relatively large variations of battery voltage. However, the output of analog-to-digital converters such as ADC 25 is not invariant with battery voltage (applied to MC 22 wherein ADC 25 is located) and so a method of correction of any such variation is highly desirable.

Circuit 160 comprises a resistor 164, a zener diode 162, and an FET 170 acting as a switch. The resistor, zener diode, and switch are connected as a series circuit between V+ and V− (FIG. 1). A gate electrode 172 is coupled to a control port of MC 22. Thus, MC 22 may effectively toggle the series circuit on or off, by application of a control voltage to electrode 172. When the series circuit is on, the circuit acts as a voltage regulator supplying a substantially invariant voltage, even with variation of voltage from battery 26, at a junction 169 between the zener diode and resistor 164. Other circuits or electronic devices driven by battery 26, for producing a substantially invariant voltage, will be apparent to those skilled in the art. All such circuits and electronic devices are assumed to be comprised within the scope of the present invention.

A pair of resistors 166 and 168 are connected in series and shunt zener diode 162, the resistor pair acting as a voltage divider having an output at a junction 167 of the resistor pair. Preferable values for resistors 164, 166, and 168 are approximately 1.5 kΩ, and zener diode 162 preferably has an operating voltage of approximately 1.2 V and a “striking” current of approximately 1 mA. Thus, battery voltages greater than about 2.2 V cause zener diode 162 to strike, and generate a substantially invariant voltage of 0.6 V at junction 167. Typically, battery 26 is considered depleted if the battery voltage is lower than about 2.2 V.

The output from junction 167 is fed by an SPDT switch 176 to ADC 25. Switch 176 is controlled by MC 22, and in a first position the switch couples junction 167 to ADC 25. In a second position the switch couples the junction of R22 and R24 to the ADC, as described above with reference to FIG. 2A.

Before operation of device 10 or device 150, ADC look-up table 29 is stored in memory 23. The table comprises outputs of ADC 25 when input with the voltage from junction 167, respective voltages of battery 26, and respective multiplicative correction factors to be applied to the outputs of the ADC. (It will be understood that when battery 26 is operating at its nominal voltage, the output of ADC 25 will be substantially correct, and the correction factor in this case is 1.) Most preferably, the table allows for changes in actual value of voltage input to the ADC due to an internal resistance 174 of the ADC, the internal resistance typically being a value of approximately 150 kΩ.

To calibrate ADC 25 during operation of device 10 or 150, the micro-controller sets switch 176 to be in its first position, and sets FET 170 to conduct so that the voltage of junction 167 is input to the ADC. An output from ADC 25 is recorded by MC 22, and the micro-controller uses table 29 to determine the battery voltage, and to determine the correction factor to be applied to readings derived from ADC 25 due to a change of battery voltage from its nominal value. Most preferably, the battery voltage and the correction factor are determined using linear interpolation of values present in table 29, or by any other method known in the art. As changes in battery voltage occur, MC 22 uses the new values of battery voltage and the correction factor for ADC 25 to ensure that the average current generated by the pulses, described above with reference to FIGS. 4 and 5, is substantially constant.

The calibration described hereinabove may be implemented at any convenient time, as determined by MC 22, most preferably when there is no requirement for device 10 or device 150 to generate stimulation pulses. Because of the generally slow rate of change of battery voltage with time, such calibrations will normally only need to be implemented relatively infrequently, such as at 24 hourly intervals. Such a regular calibration time is preferably programmed into MC 22 at installation of device 10 or 150. Most preferably, MC 22 monitors changes in battery voltage as determined by the calibration, and is adapted to change the times of calibration in the event of any relatively sudden change in battery voltage, such as a relatively sharp voltage decrease. It will be appreciated that the calibration system described hereinabove allows for measuring the voltage output from battery 26, as well as correcting for inaccuracies in ADC 25 output due to changes in the battery output voltage from a nominal output value. Measurements of the voltage output of battery 26 enable determinations of the remaining lifetime of the battery, as well as providing an indication of when the battery is near the end of its life.

FIGS. 8A and 8B are electronic diagrams of an alternative circuit 200 comprised in device 10 (FIG. 1), according to preferred embodiments of the present invention. Circuit 200 comprises a charging circuitry section 202, (FIG. 8A) and a current controlled stimulation section 204 (FIG. 8B). Apart from the differences described below, charging circuitry section 202 is generally similar to that of the AC elements of charging circuitry section 13 (FIG. 2A), such that elements indicated by the same reference numerals in both sections 13 and 202 are generally identical in construction and in operation. Elements R21, C13, C14, L1, C16, R22, and R24 in section 13 correspond respectively to R42, C19, C20, L2, R43, and R45 in section 202. A pair of capacitors C21 and C22 act as stimulation capacitors, corresponding to stimulation capacitor C15 (FIG. 2A). In contrast to capacitor C15 which is connected to ground, a first side of stimulation capacitors C21 and C22 is connected to the positive supply of battery 26, so that components in section 204 have sufficient operating voltage.

Section 202 charges stimulation capacitors C21 and C22 generally as described for the AC mode operation of section 13. However, rather than pulses STIM_SW having approximately equal on and off times, MC 22 is configured to vary the off time according to a look-up table 206 in memory 23. Table 206 comprises values of VCAP_DIV, i.e., the voltage measured by the resistance divider formed by R43 and R45, and corresponding to the charged voltage VCAP of capacitors C21 and C22. Table 206 gives “off” times for STIM_SW for different values of VCAP_DIV. MC 22 receives VCAP_DIV as an input, and uses the table to find the off time for STIM_SW.

Values for table 206 are most preferably determined experimentally, or alternatively by simulation of circuit 200, prior to installation of device 10 in subject 24. Typical off times are in a range from approximately 20 μs to approximately 50 μs to generate voltages up to approximately 25 V. By implementing variable off times for STIM_SW, the inventors have found that charge times for stimulation capacitors C21 and C22 are substantially reduced compared to having the off time fixed. It will be appreciated that reduced charge times for the stimulation capacitors means that operations of MC 22 may be substantially reduced during times when the capacitors are not being charged, leading to further overall improvements in efficiency of device 10.

In section 204 (FIG. 8B) complementary npn transistors Q6A, Q6B, and pnp transistors Q3A, Q3B, typically BC847 and BC857 transistors produced by Philips Semiconductors of Eindhoven, The Netherlands, are used to develop stimulation levels for electrodes 28 and 30. Section 204 receives VCAP at the emitter of Q3A. MC 22 generates pulse width modulated pulses CURRENT_PWM, having a variable duty cycle. The CURRENT_PWM pulses are filtered in a low pass filter comprised of R32 and C18, generating a substantially constant voltage which is input to the base of Q6A. The higher the duty cycle of CURRENT_PWM, the higher the voltage input to Q6A, and thus the higher the current through Q6A, providing a level PULSE is set to ground. If PULSE is set high, current through Q6A effectively reduces to zero. Thus, PULSE acts as a switching level for section 204, and CURRENT_PWM acts to set the current level of the section when the section is supplying current.

The current from Q6A is input to the base of Q3A, which multiplies the base current, so that Q6A and Q3A together act as a current source. The current source is activated by setting PULSE to ground, whereupon stimulation current is injected into tissue 20 via J11 through electrode 28. The stimulation current returns through electrode 30 via J9.

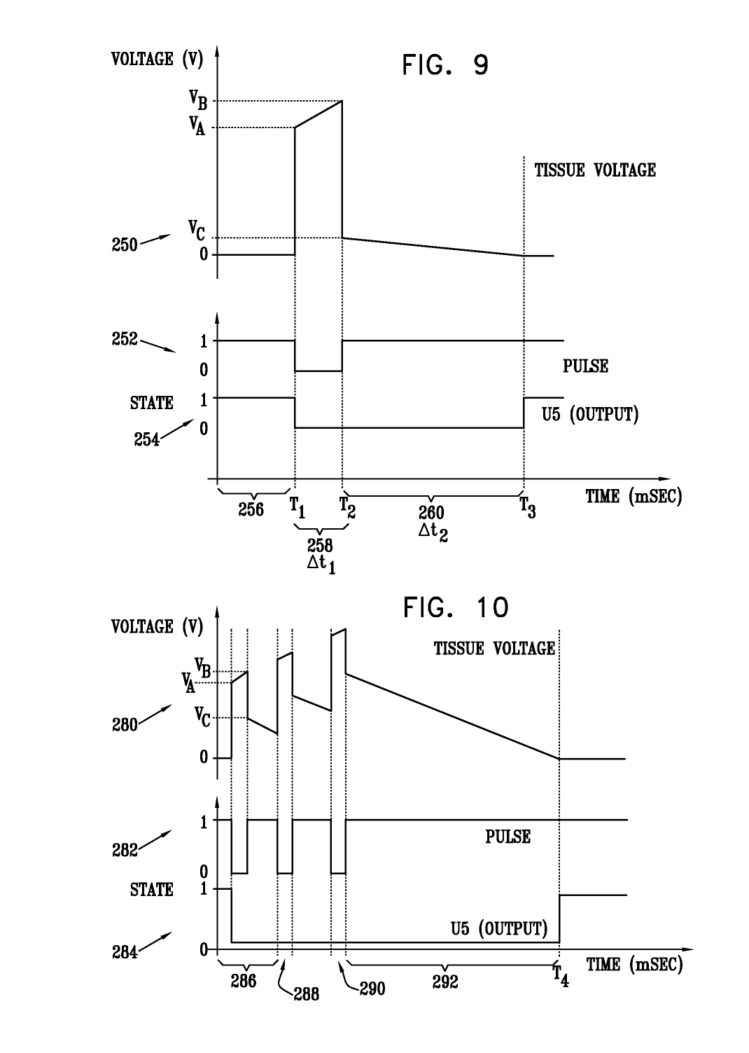

When Q3A is active, i.e., is conveying stimulation current through J11, V_{CE}, the potential between the emitter and collector of Q3A, has a pre-determined value. A transistor Q4B, preferably a field effect transistor (FET) Si1539 produced by Vishay Siliconix, acts as a detector to monitor V_{CE}. Q4B preferably has its source coupled to the emitter of Q3A, and its gate to the collector of Q3A. The drain of Q4B is coupled, via a voltage divider formed by R33 and R36, to ground. If V_{CE} is higher than a threshold voltage of Q4B, then Q4B conducts and a signal DC_SUFFICIENT, from the voltage divider formed by R33 and R36, is generated. MC 22 uses DC_SUFFICIENT to monitor and set the voltage VCAP developed by section 202, as described in more detail below with respect to FIG. 11.

A discharge phase of section 204 is activated when PULSE is set high. During this phase discharge is implemented by setting VREG, into the base of transistor Q6B, high. Typically, VREG is connected to V+. Alternatively, VREG may be connected to the filtered value of CURRENT_PWM. Q6B then passes a current having a value dependent on R41, and on the potential developed by the filtered value of CURRENT_PWM if VREG is connected thereto. The current passed by Q6B is injected to the base of Q3B where it is multiplied to form a discharge current between electrodes 28 and 30. The discharge current, opposite in direction to the stimulation current, effectively discharges interface capacitance C1(f) of tissue 20. The value of R41 is most preferably set so that the discharge current is significantly less than the charge current, typically by a factor approximately equal to ten or twenty. R41 in section 204 is, by way of example, 390 kΩ. However, R41 may be set to any other suitable fixed value, or may be implemented as an adjustable resistor, or as a resistor having a value that may be programmed by MC 22. The discharge provided by section 204 is an “active” discharge mechanism which is substantially independent of any charge on C1(f), or of the voltage between electrodes 28 and 30. Advantages of this active discharge are described in more detail below.

Discharge continues until the output of a comparator U5, preferably a MAX920 produced by Maxim Integrated Products, Inc., of Sunnyvale, Calif., goes high. U5 is configured so that its output remains low while there is a substantially non-zero potential at its input, i.e., while there is a substantially non-zero potential across capacitance C1(f), and so that its output goes high when the potential across the capacitor is substantially equal to zero. It will be appreciated that U5 comprises a feedback loop that governs the discharge of capacitance C1(f).

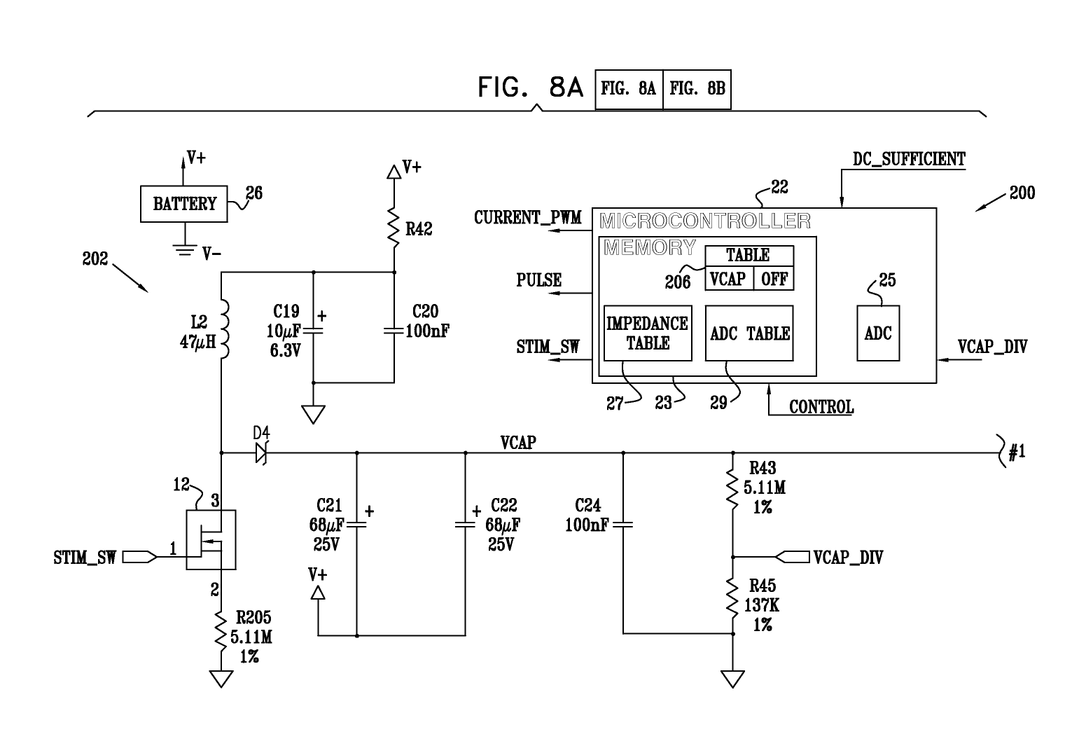

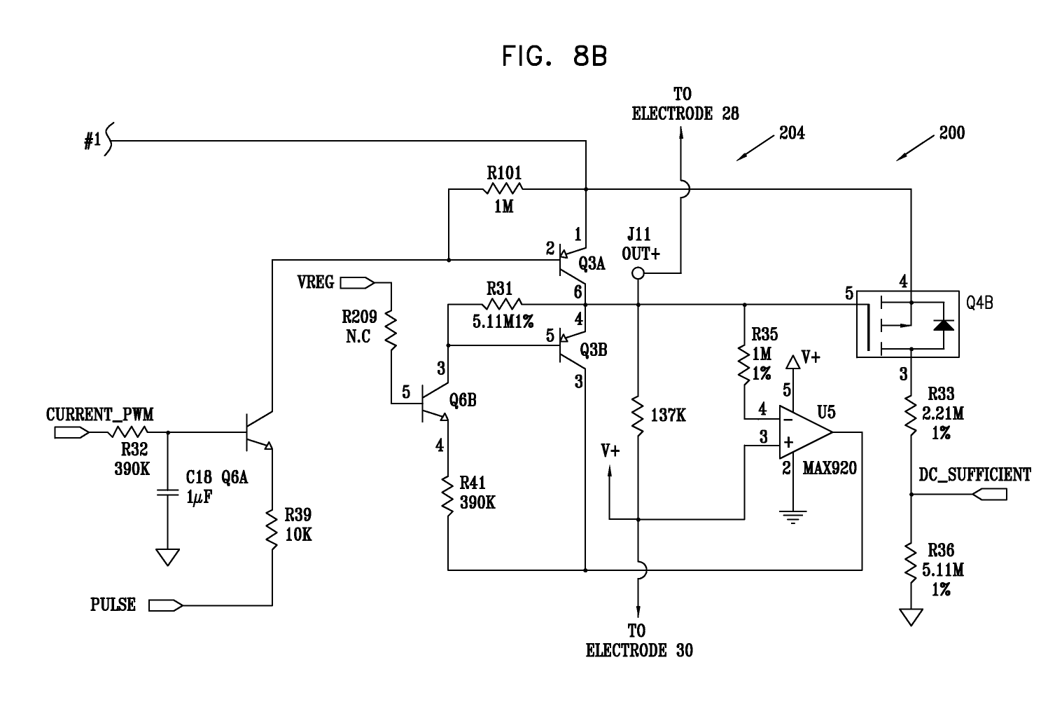

FIGS. 9 and 10 are voltage vs. time graphs illustrating operation of circuit 200. A graph 250 shows the voltage between electrodes 28 and 30, and graphs 252 and 254 respectively show voltages of PULSE and the output of U5. In a period 256, PULSE is set high, so that there is no charge current to electrodes 28 and 30, and thus the potential between the electrodes is zero. At a time T1, MC 22 sets PULSE to be low and initiates pulses CURRENT_PWM, causing charge to flow between the electrodes at a rate set by the duty cycle of CURRENT_PWM. The voltage between the electrodes thus rises to an initial value VA, typically approximately 4 V, and the output of U5 consequently goes low. During a period 258, also herein termed Δt₁ and typically having a value of approximately 1 ms, PULSE remains low, so that charge continues to flow between electrodes 28 and 30 and the voltage between the electrodes rises further, in a substantially linear manner, to a value VB, typically approximately 4.2 V. It will be appreciated that the slope of the graph in period 258 is directly proportional to the stimulation capacitance formed by C21 and C22, and to the stimulation current generated by Q3A.

At a time T2 MC 22 sets PULSE high, so that charge no longer flows to the electrodes, and so that the voltage across the electrodes falls to a value VC, typically approximately 0.2 V. This non-zero voltage maintains the output of U5 low, so that discharge of C1(f) occurs via Q3B. The discharge lowers the voltage across the electrodes during a period 260, also herein termed Δt₂ and typically having a value of approximately 10 ms, in a substantially linear manner. The discharge continues until the voltage across the electrodes, detected by U5, is zero, at a time T3. At time T3 the output of U5 thus goes high, cutting off further discharge of C1(f).

It will be appreciated that the slope of the graph in period 260, generated by the active discharge mechanism described hereinabove, is directly proportional to the stimulation capacitance and to the discharge current generated by Q3B, so that the discharge current is a substantially fixed fraction of the stimulation current. Also, the active discharge mechanism ensures that C1(f) is substantially completely discharged, regardless of the dependence of R1, R2, and C1(f) on parameters described above with reference to FIG. 1, by effectively monitoring the charge on the interface capacitance during discharge. The active discharge mechanism provides a substantially linear voltage-time discharge of the interface capacitance, in contrast to “passive” discharge systems which use a resistance, and which exponentially reduce their rate of discharge as the charge on the capacitance reduces.

Furthermore, the inventors have found that application of the active discharge mechanism, as exemplified above, to actively limit the discharge current to a maximum value, typically approximately 5% of the stimulation current, substantially eliminates anodal break excitation.

C1(f) may be calculated from the measured value of Δt₂. During discharge of C1(f), i.e., in period 260, the total charge Q delivered to the interface capacitor is given by:

Q=C1(f)·V_{S} (1)

where V_{S} is the voltage on the interface capacitor during discharge.

Also,

Q=I·Δt₂ (2)

where I is the discharge current to the interface capacitor from Q3B.

Combining equations (1) and (2) gives:

$$C 1 ( f ) = \frac{{V}_{S}}{I \cdot Δ {t}_{2}} \qquad ( 3 )$$

Inspection of equation (3) shows that the interface capacitance C1(f) is inversely proportional to Δt₂, i.e., the time of discharge, and that C1(f) may be calculated from the values of Δt₂, I, and V_{S}. A method for determining V_{S} is described with reference to equation (4) below.

As stated above, the value of interface capacitance C1(f) is a function of frequency f applied to tissue 20. Since f is inversely proportional to pulse width Δt₁, the value of C1(f) varies as Δt₁ varies. Thus, by charging the interface capacitor for different times Δt₁, and measuring the discharge time of Δt₂ in each case, the value of C1(f) for different frequencies f may be determined.

Use of equation (3), and the measurement of the variation of the interface capacitance as described above, allow for measurement of the impedance of the interface formed by electrodes 28 and 30 with tissue 20 by methods which will be apparent to those skilled in the art. Measurement of the impedance enables detection of electrode open and short circuits, for example, in cases where the electrode ruptures or moves to a new location. Such open and short circuits typically cause relatively rapid changes of impedance with time, and MC 22 may be implemented to detect the changes and alter or halt the stimulation applied by the electrodes in response to the detected changes.

Knowledge of the values of C1(f) also allows settings to be made for the charge and discharge currents that improve the overall efficiency of operation of circuit 200.

A graph 280 (FIG. 10) shows the voltage between electrodes 28 and 30, and graphs 282 and 284 respectively show voltages of PULSE and the output of U5 for an alternative type of stimulation that may be provided by circuit 200. A time period 286 corresponds to periods 256, 258 and the first part of 260 (FIG. 9). In periods 288 and 290, however, MC 22 sets PULSE to be low and operates pulses CURRENT_PWM, implementing the charging cycle before the voltage across electrodes 28 and 30 has reduced to zero. In a period 292, discharge is allowed between the electrodes, substantially as described above for period 260, until at a time T4 the voltage across the electrodes becomes zero, whereupon the output of U5 goes high. Typically a time between periods 288 and 290 is approximately 5 ms, and period 292 is approximately 20 ms.

It will be appreciated that stimulation pulses other than those exemplified by FIG. 9 and FIG. 10 may be generated by circuit 200, using alternative times for PULSE to be high and low. All such stimulation pulses are assumed to be comprised within the scope of the present invention.

In operating circuit 200, high efficiency is achieved by maintaining stimulation capacitors C21 and C22 at a minimum potential sufficient to drive section 204. Preferred embodiments of the present invention use DC_SUFFICIENT to regulate the potential of the stimulation capacitors so as to achieve such high efficiency, as is described with reference to FIG. 11 below.

FIG. 11 is a flowchart 300 showing steps involved in setting the voltage on stimulation capacitors C21 and C22, according to a preferred embodiment of the present invention. Implementing the steps of the flowchart allows circuit 200 to attempt to minimize an absolute difference between the actual voltage VCAP on the stimulation capacitor and a targeted voltage V_{REQ} for the capacitor, set by MC 22. In following the flowchart, both V_{REQ} and VCAP may vary so as to attempt to minimize the difference.

Referring back to circuit 200, the following condition holds:

VCAP=VCE+VS (4)

where VS is the stimulation potential applied between electrodes 28 and 30, and

VCE is the potential between the emitter and collector of transistor Q3A.

It will be appreciated that V_{S} may be determined from values of VCAP and V_{CE}.

During operation of circuit 200, V_{S} must be sufficient to provide current through tissue 20, and the current is controlled by transistor Q3A. In order to act as a controller, Q3A must be in a conducting state, so that MC 22 must ensure that V_{CE} must be greater than a minimum transistor operating voltage, typically approximately 0.2 V. In addition, MC 22 adjusts V_{CE} to accommodate changes in VCAP, so as to maintain V_{STIM} at the required potential. The state of Q3A is monitored by transistor Q4B, which generates DC_SUFFICIENT. DC_SUFFICIENT acts as a Boolean signal indicating whether Q3A is conducting or not. Preferably, DC_SUFFICIENT is set to be TRUE or FALSE according to the following condition:

VCE>VOP,DC_SUFFICIENT is TRUE,

VCE<VOP,DC_SUFFICIENT is FALSE (5)

where VOP is an operating potential at which Q3A is maintained.

Typically, MC 22 sets V_{OP} to be approximately 1.5 V. In the description below, V_{OP} is assumed to be 1.5 V.

MC 22 preferably implements the steps of flowchart 300 at the end of each charging period when PULSE goes high, e.g., at T2 (FIG. 9) and at the end of periods 288 and 290 (FIG. 10).

In a first comparison 306, MC 22 checks the value of DC_SUFFICIENT. If DC_SUFFICIENT is FALSE, then in a second comparison 304 MC 22 checks that VREQ was reached during the charging period. If VREQ was not reached during the charging period the flowchart ends. If VREQ was reached, then in a step 308 VREQ is incremented, typically by a value approximately equal to 0.5V. In a step 316 the value of VREQ is checked to ensure it is less than a limiting operational maximum voltage Vmax. The process of incrementing VREQ thus applies when VCAP<VS+1.5 V.

If comparison 306 returns DC_SUFFICIENT as TRUE, then VCAP>VS+1.5 V. When DC_SUFFICIENT is TRUE, in a third comparison 310 MC 22 compares the value of VCAP with VREQ, using the output VCAP_DIV. If VCAP>VREQ, then the flowchart ends. If VCAP≦VREQ, then in a step 312 MC 22 decreases VREQ, typically by approximately 0.25V. In a step 314 the value of VREQ, is checked to ensure it is greater then a limiting operational minimum voltage Vmin.

It will be appreciated that implementation of flowchart 300 leads to a system wherein MC 22 varies VCAP by varying VCE about VOP. By implementing flowchart 300, MC 22 maintains the stimulation capacitors at a potential that is only slightly greater than the minimum voltage necessary for circuit 200 to operate, leading to very efficient operation of the circuit.

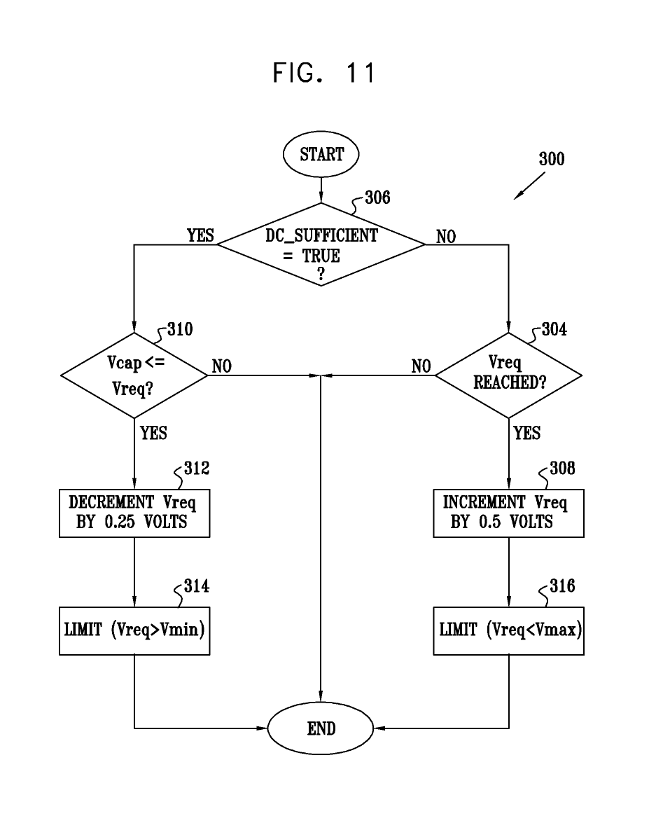

FIG. 12 shows graphs illustrating the operation of flowchart 300, according to a preferred embodiment of the present invention. The graphs are simulations of voltages vs. time when circuit 200 has reached a steady state, assuming a value of VOP=1.5 V and a value of VS=4 V. A graph 354 shows values of VREQ set by MC 22; a graph 356 shows values of VCAP on the stimulation capacitor; and a graph 358 shows values of signal DC_SUFFICIENT, a value of 5V corresponding to TRUE, and 4V corresponding to false.

MC 22 operates flowchart at times 352 and 360, corresponding to times just after stimulation has been applied, as shown by the sharp drop in values of VCAP. At a first time 352 (150 ms), DC_SUFFICIENT is TRUE and VCAP<VREQ, so that step 312 of flowchart 300 is reached, and MC 22 decrements VREQ. The same conditions hold at other times 352, so that in each case VREQ is decremented.

At a first time 360 (550 ms) DC_SUFFICIENT is FALSE and VREQ was reached during the charging period, as shown by VCAP being greater than VREQ in the immediately preceding pulse. Step 308 of flowchart 300 is thus reached, so that MC 22 increments VREQ. The same conditions hold at other times 360, in each case VREQ being incremented.

As illustrated by graph 356, operation of flowchart 300 causes VCAP to vary about a value approximately equal to 5.5 V (VOP+VS), by effectively incorporating a feedback effect between VCAP and VREQ. The feedback effect enables future values of VCAP to correspond with values of VREQ. As also illustrated by the graphs, operation of flowchart 300 causes VCAP to always be sufficient for MC 22 to generate stimulation, but never to rise above a maximum value, approximately equal to 6.3 V in the simulation shown.

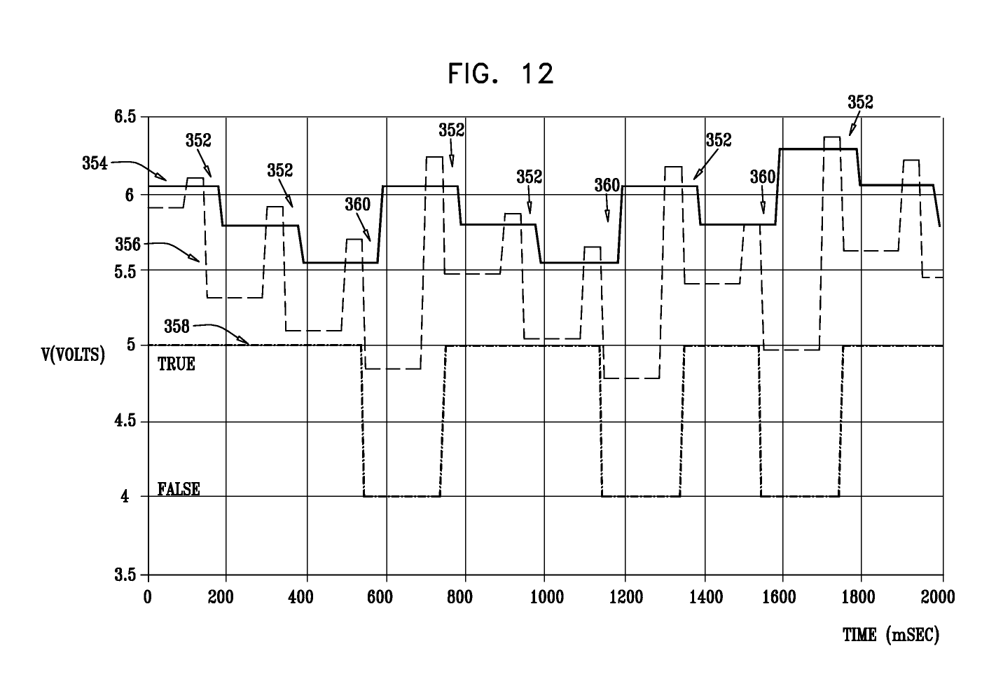

Reference is now made to FIG. 13A, which is a schematic, cross-sectional illustration of an electrode assembly 420 for applying current to a nerve 430, in accordance with an embodiment of the present invention. It is noted that although the various electrode assemblies shown in the figures and described herein generally contain cylindrical configurations of their elements, other geometrical configurations, such as non-rotationally symmetric configurations, are also suitable for applying the principles of the present invention. In particular, a housing of the electrode assemblies (and the electrodes themselves) may form a complete circle around the nerve, or it may define an arc between approximately 0 and 90 degrees, between 90 and 180 degrees, between 180 and 350 degrees, or between 350 and 359 degrees around the nerve. For some applications, the electrode assemblies shown in the figures and described herein comprise electrodes that form rings around the nerve, and an insulating, elastic cuff that surrounds the electrodes.

Electrode assembly 420 comprises at least one active, i.e., stimulating and/or sensing, electrode 438, such as at least one cathode 441 and at least one anode 442. Each of these electrodes is fixed within a housing 422 of the electrode assembly. Active electrodes 438 are coupled to an implantable or external control unit 440 by leads 443 and 444. For some applications, active electrode configurations and/or stimulation techniques are used which are described in one or more of the patent applications incorporated by reference hereinbelow.

Electrode assembly 420 further comprises two or more passive electrodes 450, fixed within housing 422, and a conducting element 452, typically a wire, which electrically couples the passive electrodes to one another. The electrode assembly is configured such that the passive electrodes are electrically device-coupled, as defined hereinabove, to neither (a) any circuitry that is electrically device-coupled to the at least one cathode 441 or the at least one anode 442, nor (b) an energy source. Passive electrodes 450 and conducting element 452 create an electrical path for current that would otherwise leak outside electrode assembly 420 and travel around the outside of the housing through tissue of the subject.

For some applications, the active electrodes are positioned within housing 422 longitudinally between the two or more passive electrodes 450 (as shown in FIG. 13A). Alternatively, at least one of the passive electrodes is positioned between the at least one cathode and the at least one anode (configuration not shown).

Internal insulating elements 424, which are either part of the body of the housing or affixed thereto, are typically placed so as to separate the electrodes, and to guide current from one of the electrodes towards the nerve prior to being taken up by another one of the electrodes. Typically (as shown), the insulating elements are closer to nerve 430 than are the electrodes. Alternatively (not shown), insulating elements 424 are generally flush with the faces of the electrodes. The electrode assembly typically further comprises one or more end insulating elements 426, which extend along nerve 430 in order to electrically isolate a portion of the nerve within housing 422 from a portion of the nerve outside the electrode assembly. The end insulating elements help direct any current that leaks from the active electrodes through the electrical path created by the passive electrodes and the conducting element. For some applications, conducting element 452 comprises at least one passive element 454, such as a resistor, capacitor, and/or inductor.

For some applications, the electrode assembly is configured to selectively stimulate fibers of the nerve having certain diameters, such as by using techniques described in one or more of the patent applications incorporated by reference hereinbelow. For example, control unit 440 may drive cathode 441 to apply to nerve 430 a stimulating current, which is capable of inducing action potentials in a first set and a second set of nerve fibers of the nerve, and drive anode 442 to apply to the nerve an inhibiting current, which is capable of inhibiting the induced action potentials traveling in the second set of nerve fibers, the nerve fibers in the second set having generally larger diameters than the nerve fibers in the first set.

For some applications, the electrode assembly is configured to apply unidirectional stimulation to the nerve, such as by using techniques described in one or more of the patent applications incorporated by reference hereinbelow. For example, control unit 440 may drive anode 442 to apply an inhibiting current capable of inhibiting device-induced action potentials traveling in a non-therapeutic direction in nerve 430. For some applications, electrode assembly 420 comprises primary and secondary anodes, the primary anode located between the secondary anode and the cathode. The secondary anode is typically adapted to apply a current with an amplitude less than about one half an amplitude of a current applied by the primary anode.

Reference is now made to FIG. 13B, which is a schematic, cross-sectional illustration of an electrode assembly 480 for applying current to nerve 430, in accordance with an embodiment of the present invention. Electrode assembly 480 comprises one or more passive electrodes 450 which are not electrically device-coupled to one another. For some applications, the electrode device comprises exactly one passive electrode 450. A separate conducting element 482, typically a wire 484, is coupled to each passive electrode at a first end of the conducting element. The second end of the conducting element terminates at a relatively-remote location in the body of the subject that is at a distance of at least 1 cm, e.g., at least 2 or 3 cm, from electrode assembly 480. The remote location in the body thus serves as a ground for the passive electrode. For some applications, a remote electrode 492 is coupled to the remote end of the conducting element, so as to increase electrical contact with tissue at the remote location 490. For some applications, remote electrode 492 is configured for insertion into muscle tissue 494 of the body, in order to provide better anchoring of the second end of the conducting element at the remote location, and/or better grounding for the passive electrode.

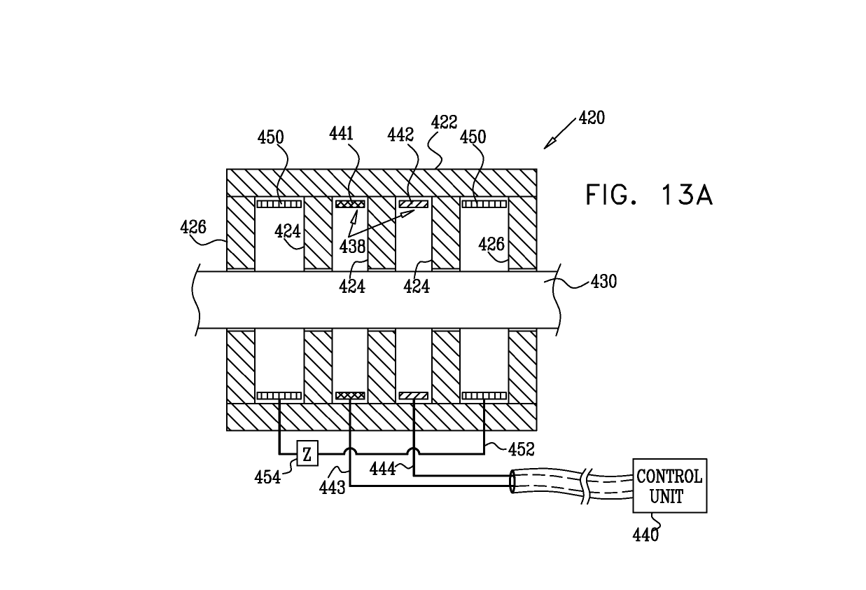

Reference is made to FIG. 14, which is a schematic, cross-sectional illustration of an electrode assembly 520 for applying current to nerve 430, in accordance with an embodiment of the present invention. Electrode assembly 520 comprises two cathodes 541a and 541b and at least one anode 542, which are fixed within a housing 522 such that no anodes are positioned between the two cathodes. Cathodes 541a and 541b are electrically coupled to one another, and are coupled to an implantable or external control unit 540 by a lead 543. Anode 542 is coupled to control unit 540 by a lead 544. Typically, a closest distance D between the two cathodes (i.e., the distance between the respective cathodes' edges that are closest to one another) is equal to at least a radius R of nerve 430, e.g., at least 1.5 times the radius of the nerve.

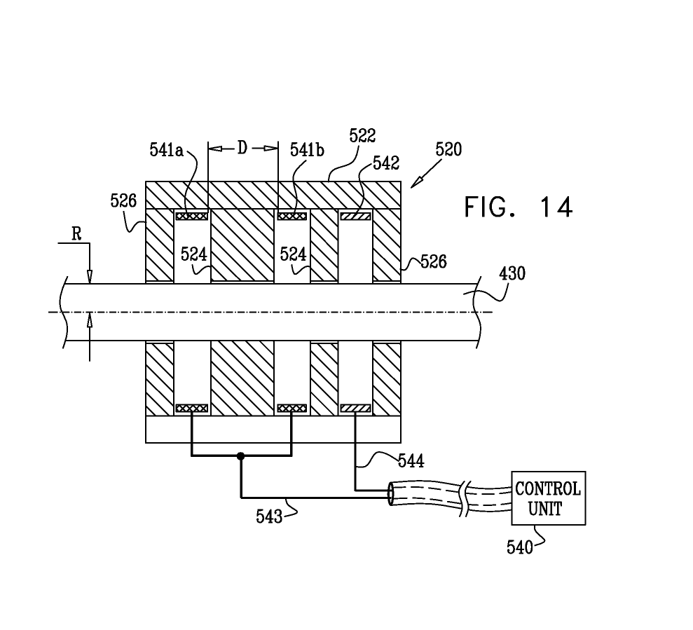

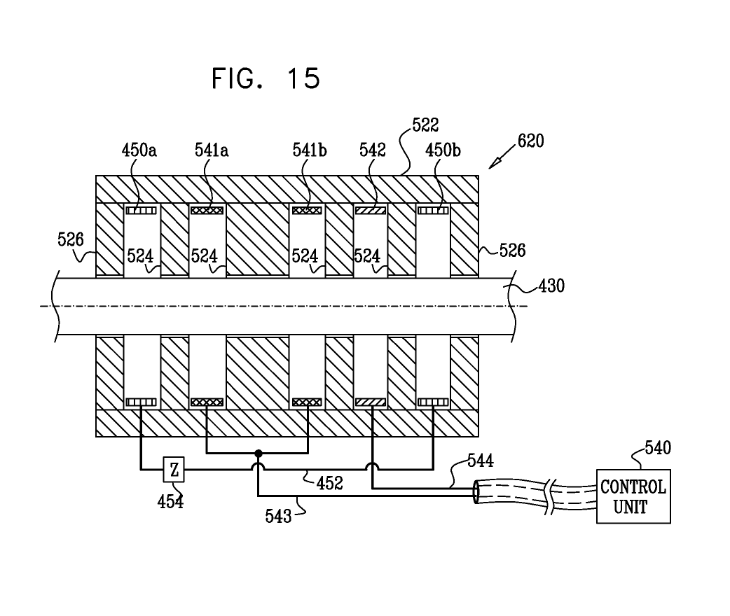

As described in detail hereinbelow with reference to FIGS. 16 and 17, this electrode configuration creates a combined cathode having an activation function a peak of which has a magnitude less than that of anode 542, which results in a stimulation that results in unidirectional propagation of action potentials in the nerve, in the direction going from anode 542 towards the cathodes. Typically, this electrode configuration also creates a virtual anode on the side of the cathodes opposite that of the anode, which results in selective fiber stimulation of fibers of the nerve having relatively small diameters.

Typically, electrode assembly 520 does not comprise any anodes on the side of the cathodes opposite anode 542 (i.e., the left side in the figures). However, for some applications, in which the virtual anode created on the side of the cathodes opposite the anode is not strong enough to create sufficient selective fiber stimulation, electrode assembly 520 comprises a second anode on the side of cathodes 541a and 541b opposite anode 542. A portion of the anodal current is driven through this anode in order to strengthen the blocking of larger-diameter fibers, thereby increasing the selection of the stimulation of small-diameter fibers. Typically, only a relatively small portion of the anodal current is driven through this second anode, in order to leave sufficient current for anode 542 to block all (or a very large portion of) action potentials generated by cathodes 541a and 541b (i.e., in order to preserve unidirectional stimulation).

For some applications, the electrode configuration of electrode assembly 520 is combined with electrode configurations and/or stimulation techniques described in one or more of the patent applications incorporated by reference hereinbelow.

Internal insulating elements 524, which are either part of the body of the housing or affixed thereto, are typically placed so as to separate the electrodes, and to guide current from one of the electrodes towards the nerve prior to being taken up by another one of the electrodes. Typically (as shown), the insulating elements are closer to nerve 430 than are the electrodes. Alternatively (not shown), insulating elements 524 are generally flush with the faces of the electrodes. The electrode assembly typically further comprises one or more end insulating elements 526, which extend along nerve 430 in order to electrically isolate a portion of the nerve within housing 522 from a portion of the nerve outside the electrode assembly.

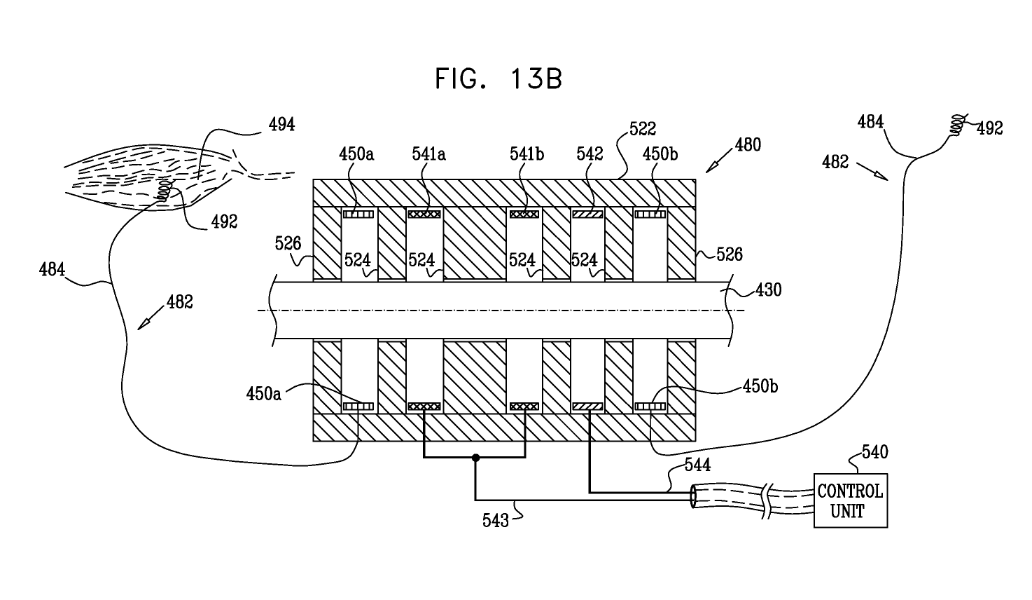

Reference is made to FIG. 15, which is a schematic, cross-sectional illustration of an electrode assembly 620 for applying current to nerve 430, in accordance with an embodiment of the present invention. Electrode assembly 620 is the same as electrode assembly 520, described hereinabove with reference to FIG. 14, except that electrode assembly 620 further comprises, on the ends thereof, two passive electrodes 450a and 450b and conducting element 452, as described hereinabove with reference to FIG. 13A. Typically, a closest distance between anode 542 and passive electrode 450b is between about 0.7 mm and about 1 mm. Conducting element 452 optionally comprises passive element 454, as described hereinabove with reference to FIG. 13A.

Reference is now made to FIGS. 16 and 17, which are graphs modeling calculated activation functions 400 and 402, respectively, of myelinated nerve fibers having a diameter of 1 micrometer, over a portion of the length of nerve 430, when current is applied using an electrode assembly similar to that shown in FIG. 15, in accordance with an embodiment of the present invention. For the purposes of modeling these activation functions, (a) cathodes 541a and 541b are placed at longitudinal sites on the nerve labeled z=−2 mm and z=2 mm, respectively, (b) anode 542 is placed at a longitudinal site z=4.1 mm, and (c) passive electrodes 450a and 450b are placed at longitudinal sites z=−4.1 mm and z=5.5 mm, respectively. All of the electrodes are placed at a radius of 2.5 mm from the axis of nerve 430, which has a radius of 1.35 mm. Activation functions 400 (FIG. 16) and 402 (FIG. 17) are modeled at radii of 1.2 mm from the axis of nerve 430 (near the surface of the nerve) and 0 mm (i.e., at the axis of the nerve), respectively.

Activation function 400 (FIG. 16) has two depolarization peaks 404 and 406, at approximately z=−2.5 and z=2.5, corresponding to the longitudinal positions of the two cathodes. In activation function 402 (FIG. 17), these two depolarization peaks have partially combined into a single, wide depolarization peak 408. Each of activation functions 400 and 402 has a hyperpolarization peak 410 at approximately z=4, corresponding to the longitudinal position of the anode. For a given fiber diameter (in this case, 1 micrometer), at all depths within the nerve, the amplitude of the hyperpolarization peak is greater than the amplitude of greatest depolarization peak, such as at least 10% or 20% greater. As a result, the hyperpolarization peak blocks propagation of substantially all cathode-induced action potentials traveling in the nerve from the cathode in the direction of the anode (i.e., to the right in the figures).

Each of activation functions 400 and 402 has a second, smaller hyperpolarization peak 412 at between about z=−4 and about z=−5, approximately corresponding to the longitudinal position of passive electrode 450a. This “virtual anode” effect, which is caused by cathodes 541a and 541b and passive electrode 450a, blocks propagation of almost all cathode-induced action potentials traveling in large- and medium-diameter fibers, but not those in small-diameter fibers, resulting in selective small-diameter fiber activation in the direction from cathode 541a to passive electrode 450a (i.e., to the left in the figures). It is noted that in the absence of passive electrode 450a (in the embodiment described with reference to FIG. 14), the two-cathode configuration still results in the virtual anode effect between about z=−4 and about z=−5. Current flows through tissue around the outside of the electrode assembly, rather than between the passive electrodes via conducting element 452.

Activation functions 400 and 402 additionally have a second, smaller depolarization peak 414 at between about z=6 and z=8, approximately corresponding to the longitudinal position of passive electrode 450b. This “virtual cathode” effect, which is caused by anode 542 and passive electrode 450b, does not generate action potentials in more than 10% of axons of nerve 430 (or does not generate action potentials in any axons of nerve 430) because of the virtual cathode's relatively low amplitude, and its vicinity to strong hyperpolarization peak 410.

It is noted that if cathodes 541a and 541b are positioned at a closest distance D less than radius R of nerve 430 (FIG. 14), the cathodes begin to behave as a single cathode, generating a depolarization peak having a greater amplitude than those in activation functions 400 and 402. As a result, the amplitude of hyperpolarization peak 410 is no longer greater than the amplitude of the greatest depolarization peak at all nerve fiber diameters. The stimulation is thus not unidirectional at all nerve fiber diameters.

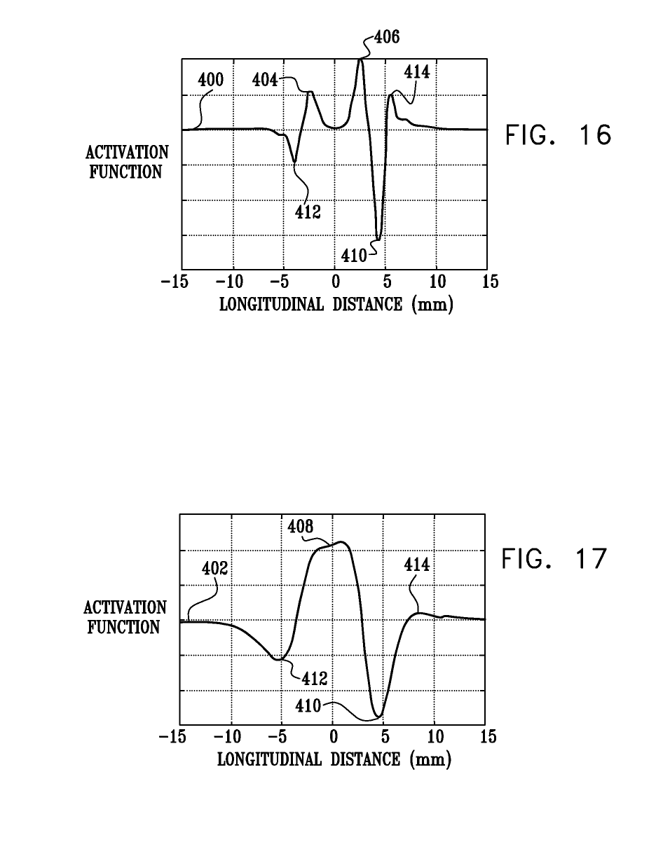

Reference is made to FIG. 18, which is a schematic, cross-sectional illustration of an electrode assembly 720 for applying current to nerve 430, in accordance with an embodiment of the present invention. Electrode assembly 720 comprises one or more electrodes, such as at least one cathode 741 and at least one anode 742, which are fixed within a housing 722. Electrode assembly 720 further comprises two elongated end insulating elements 726, which are either part of the body of the housing or affixed thereto. The end insulating elements extend along nerve 430 in order to electrically isolate a portion of the nerve within housing 722 from a portion of the nerve and other tissue outside the electrode assembly. Each of the end insulating elements has a length L of at least 2 mm, such as at least 3 mm, or at least 4 mm. This elongation of the end insulating elements tends to lengthen the electrical path around the outside of the electrode assembly through tissue of the subject, thereby reducing the current that leaks from the assembly and flows through this path.

For some applications, the insulating elements have a thickness T along at least 75% of their length of less than about 0.5 mm. For some applications, at least one internal insulating element 724, which is either part of the body of the housing or affixed thereto, is placed so as to separate the electrodes, and to guide current from one of the electrodes towards the nerve prior to being taken up by another one of the electrodes.

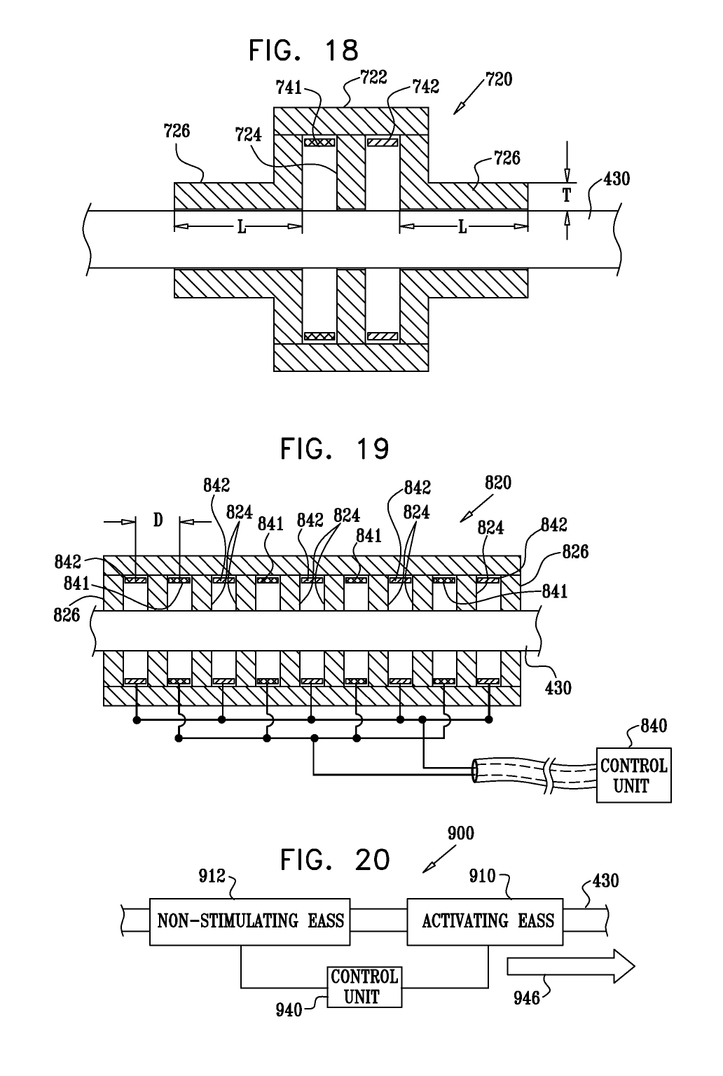

Reference is made to FIG. 19, which is a schematic, cross-sectional illustration of an electrode assembly 820 for applying Electrode Array Selective Stimulation (EASS) to nerve 430, in accordance with an embodiment of the present invention. EASS assembly 820 comprises alternating anodes 842 and cathodes 841. Typically, five anodes 842 and four cathodes 841 are adequate to create a periodic electric field along a sufficiently long length of nerve. A control unit 840 is configured to drive the electrodes of EASS assembly 820 to apply a spatially-periodic field to nerve 430 that is configured to target fibers of a selected diameter, as described in the Background of the Invention section hereinabove.

Internal insulating elements 824, which are either part of the body of the housing or affixed thereto, are typically placed so as to separate the electrodes, and to guide current from one of the electrodes towards the nerve prior to being taken up by another one of the electrodes. Typically (as shown), the insulating elements are closer to nerve 430 than are the electrodes. Alternatively (not shown), insulating elements 824 are generally flush with the faces of the electrodes. The electrode assembly typically further comprises one or more end insulating elements 826, which extend along nerve 430 in order to electrically isolate a portion of the nerve within the housing from a portion of the nerve outside the electrode assembly.

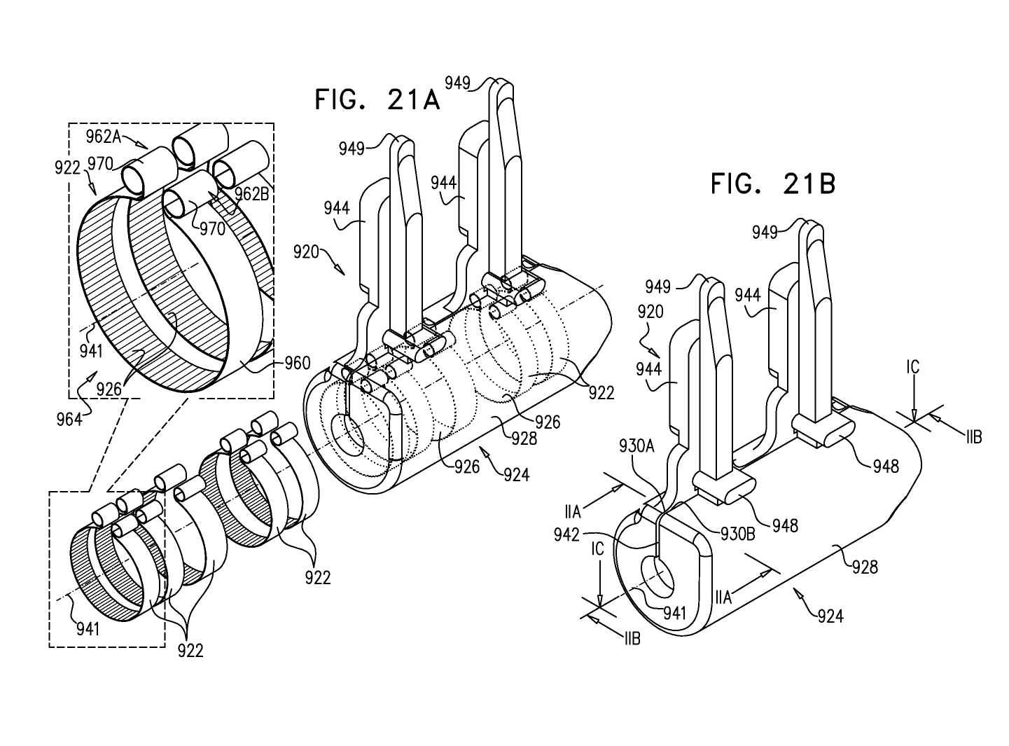

Reference is made to FIG. 20, which is a schematic illustration of a selective stimulation EASS system 900, in accordance with an embodiment of the present invention. EASS system 900 comprises two EASS assemblies 820 (FIG. 19), and a control unit 940 electrically coupled to both assemblies. The control unit, at any given time, configures one of the assemblies to function as an activating EASS assembly 910, and the other to function as a non-stimulating EASS assembly 912. (Alternatively, one of the assemblies is permanently configured to function as activating EASS assembly 910, and the other is permanently configured to function as non-stimulating EASS assembly 912.)

Control unit 940 drives activating EASS assembly 910 to apply a spatially-periodic stimulating field to nerve 430, and configures the field to induce, in small fibers of the nerve, action potentials that propagate towards a target site (in the direction indicated by an arrow 946) and away from the target site. The control unit also drives non-stimulating EASS assembly 912 to apply a spatially-periodic non-stimulating field, and to configure the field to partially depolarize the small fibers of the nerve, without initiating action potentials therein or in larger fibers of the nerve. The partial depolarization of the small fibers is sufficient to inhibit the action potentials generated by activating EASS assembly 910 in a direction opposite the target site from continuing to propagate beyond the inhibition site of non-stimulating EASS assembly 912. As a result, unidirectional small-diameter fiber stimulation is achieved towards the target site (i.e., in the direction indicated by arrow 946). Both the stimulating and non-stimulating fields are typically applied using short pulses, e.g., pulses having a duration of between about 10 and about 100 microseconds. The amplitude of the stimulating pulses is typically between about 0.5 and about 15 mA, depending on the number of fibers to be activated, and the amplitude of the non-stimulating pulses is typically between about 0.1 and about 5 mA, depending on the number of fibers to be blocked. During each application of stimulation, the non-stimulating field is typically applied slightly before application of the stimulating field, e.g., between about 500 and about 0 microseconds earlier.

Application of the non-stimulating field by non-stimulating EASS assembly 912 causes a partial depolarization of the target axons, which causes some of the ion channels in the axons to begin their gating cycles. However, the non-stimulating field is configured so to minimize the likelihood of causing depolarization sufficient to trigger an action potential. As a result of the partial depolarization, a portion of the ion channels enter their refractory periods. When a stimulating field is subsequently applied, these channels cannot begin the gating cycle. As a result, the number of channels available is insufficient to trigger an action potential. The fibers are therefore unable to transmit action potentials.

Typically, the field applied by non-stimulating EASS assembly 912 is configured to partially depolarize only small fibers, by using the EASS selective-fiber-diameter stimulation techniques described hereinabove.

It is noted that the triggering thresholds of axons vary based on the axons' diameters. Thus, a pulse of a magnitude sufficient to partially depolarize small fibers may trigger action potentials in large-diameter fibers. Therefore, without the use of the EASS partial depolarization techniques described herein, application of a conventional depolarizing pulse causes undesired complete depolarization (i.e., action potential generation) in large-diameter fibers, in addition to the desired partial depolarization of small-diameter fibers.

Alternatively, for some applications, applying the non-stimulating field comprises applying a non-EASS non-stimulating pulse, at a strength sufficient to cause partial depolarization of target axons, but insufficient to trigger an action potential.

For some applications, EASS system 900 comprises a single long EASS electrode assembly. The non-stimulating pulse is applied by a portion (e.g., about half) of the electrode assembly on the side thereof further from the target direction. The stimulating pulse is applied by the remaining portion (e.g., the other half) of the electrode assembly on the side thereof closer to the target direction. Alternatively, the stimulating pulse is applied by the entire electrode assembly, or any sufficiently long portion thereof. Use of the entire electrode device for applying the stimulating pulse generally results in a more periodic field having a lower current density.

In an embodiment of the present invention, EASS system 900 is configured to perform unidirectional stimulation of a human vagus nerve. The system is configured to selectively activate only A-delta fibers, while not activating A fibers. In the human vagus nerve, the conduction velocity of A and A-delta fibers is about 20 msec and about 8 msec, respectively (see Evans M S et al., cited hereinabove). These velocities imply nodal gaps of 0.5 mm and 0.2 mm, respectively. The inter-electrode distance D between the respective centers of the electrodes (FIG. 19) is thus typically about 0.5 mm, which enables excitation of A-delta fibers up to a depth from the nerve surface of approximately 0.5 mm. For a human vagus nerve with a diameter of about 2.5 mm, these fibers constitute about 75% of the fibers in the bundle, assuming that they are uniformly scattered throughout the bundle. The length of a single EASS electrode with five anodes and four cathodes (as shown in FIG. 19) is typically less than about 1 cm, and two such electrodes are readily placed side-by-side on a human vagus nerve.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. Provisional Patent Application 60/383,157 to Ayal et al., filed May 23, 2002, entitled, “Inverse recruitment for autonomic nerve systems,”
International Patent Application PCT/IL02/00068 to Cohen et al., filed Jan. 23, 2002, entitled, “Treatment of disorders by unidirectional nerve stimulation,” and U.S. patent application Ser. No. 10/488,334, in the national stage thereof, which issued as U.S. Pat. No. 7,734,355,
U.S. patent application Ser. No. 09/944,913 to Cohen and Gross, filed Aug. 31, 2001, entitled, “Treatment of disorders by unidirectional nerve stimulation,” which issued as U.S. Pat. No. 6,684,105,
U.S. patent application Ser. No. 09/824,682 to Cohen and Ayal, filed Apr. 4, 2001, entitled “Method and apparatus for selective control of nerve fibers,” which issued as U.S. Pat. No. 6,600,954,
U.S. patent application Ser. No. 10/205,475 to Gross et al., filed Jul. 24, 2002, entitled, “Selective nerve fiber stimulation for treating heart conditions,” which issued as U.S. Pat. No. 7,778,703,
U.S. patent application Ser. No. 10/205,474 to Gross et al., filed Jul. 24, 2002, entitled, “Electrode assembly for nerve control,” which issued as U.S. Pat. No. 6,907,295,
International Patent Application PCT/IL03/00431 to Ayal et al., filed May 23, 2003, entitled, “Selective nerve fiber stimulation for treating heart conditions,” which published as PCT Publication WO 03/099377,
International Patent Application PCT/IL03/00430 to Ayal et al., filed May 23, 2003, entitled, “Electrode assembly for nerve control,” and U.S. patent application Ser. No. 10/529,149, in the national stage thereof, which published as US Patent Application Publication 2006/0116739,
U.S. patent application Ser. No. 10/719,659 to Ben David et al., filed Nov. 20, 2003, entitled, “Selective nerve fiber stimulation for treating heart conditions,” which issued as U.S. Pat. No. 7,778,711,
U.S. patent application Ser. No. 11/022,011 to Cohen et al., filed Dec. 22, 2004, entitled, “Construction of electrode assembly for nerve control,” which issued as U.S. Pat. No. 7,561,922, and
U.S. patent application Ser. No. 11/234,877 to Ben-David et al., filed Sep. 22, 2005, entitled, “Selective nerve fiber stimulation,” which issued as U.S. Pat. No. 7,885,709.

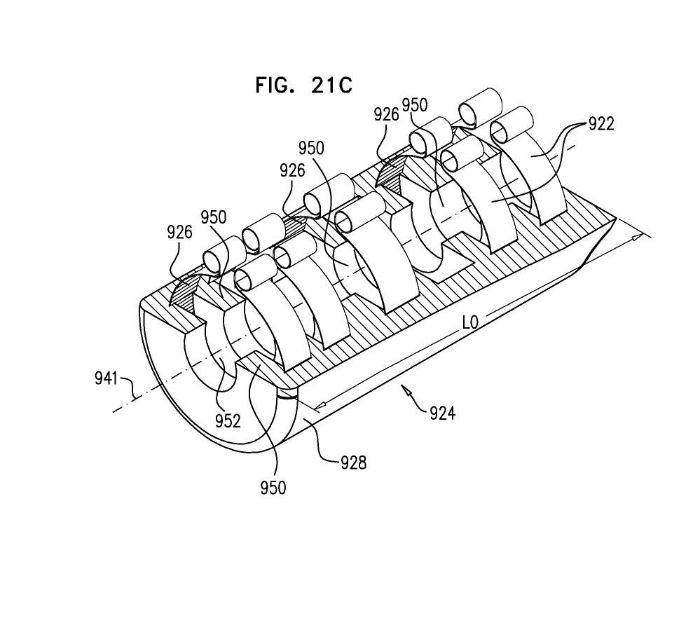

Reference is now made to FIGS. 21A-C, which are schematic illustrations of an electrode assembly 920, in accordance with an application of the present invention. Electrode assembly 920 comprises a cuff 924 and one or more electrodes 922. For clarity of illustration, electrodes 922 are shown twice in FIG. 21A, both removed from cuff 924 (at the left side of the figure) and disposed within the cuff; in actual practice, the electrodes are disposed within the cuff, as shown at the right side of the figure and in the other figures. FIG. 21C is a cut-away illustration of electrode assembly 920, in which a portion of cuff 924 is not shown in order to better illustrate electrodes 922. Electrodes 922 are fixed to cuff 924 such that the electrodes define respective exposed surfaces 926 facing axis 941.

Cuff 924 is shaped so as to define a tubular housing 928 that defines and at least partially surrounds (typically entirely surrounds) a longitudinal axis 941 therealong. Cuff 924 is configured to be placed at least partially around (typically entirely around) a nerve or other tubular body tissue, such as a blood vessel, a muscle, a tendon, a ligament, an esophagus, intestine, a fallopian tube, a neck of a gall bladder, a cystic duct, a hepatic duct, a common hepatic duct, a bile duct, and/or a common bile duct. Alternatively or additionally, cuff 924 is configured to be placed at least partially around (typically entirely around) an elliptical (e.g., circular) cylinder, which, for example, may have a major axis that is between 1 and 8 mm and a minor axis that is between 0.5 and 6 mm. As used in the present application, including in the claims, “tubular” means having the form of an elongated hollow object that defines a conduit therethrough. A “tubular” structure may have varied cross-sections therealong, and the cross-sections are not necessarily circular. For example, one or more of the cross-sections may be generally circular, or generally elliptical but not circular, circular, or irregularly shaped.

Cuff 924 comprises an elastic, electrically-insulating material such as silicone or a silicone copolymer, which may have, for example, a hardness of between about 10 Shore A and about 90 Shore A, such as about 40 Shore A. Optionally, cuff 924 comprises more than one material; for example, housing 928 and insulating elements 950, described hereinbelow, may comprise different materials, e.g., may comprise silicone having different hardnesses.

Electrode assembly 920 optionally further comprises a lead assembly, which comprises one or more electrical leads, as is known in the art. For example, the lead assembly may be implemented as described in U.S. application Ser. No. 12/952,058 (which issued as U.S. Pat. No. 8,565,896), filed Nov. 22, 2010, which is assigned to the assignee of the present application and is incorporated herein by reference. The leads are coupled to all or a portion of electrodes 922. The lead assembly couples electrode assembly 920 to an implanted or external control unit, which comprises appropriate circuitry for driving current between two or more of electrodes 922, as is known in the art. Typically, the control unit configures the current such that one or more of the contact surfaces function as cathodes, and one or more function as anodes, such as described hereinbelow with reference to Tables II and III.

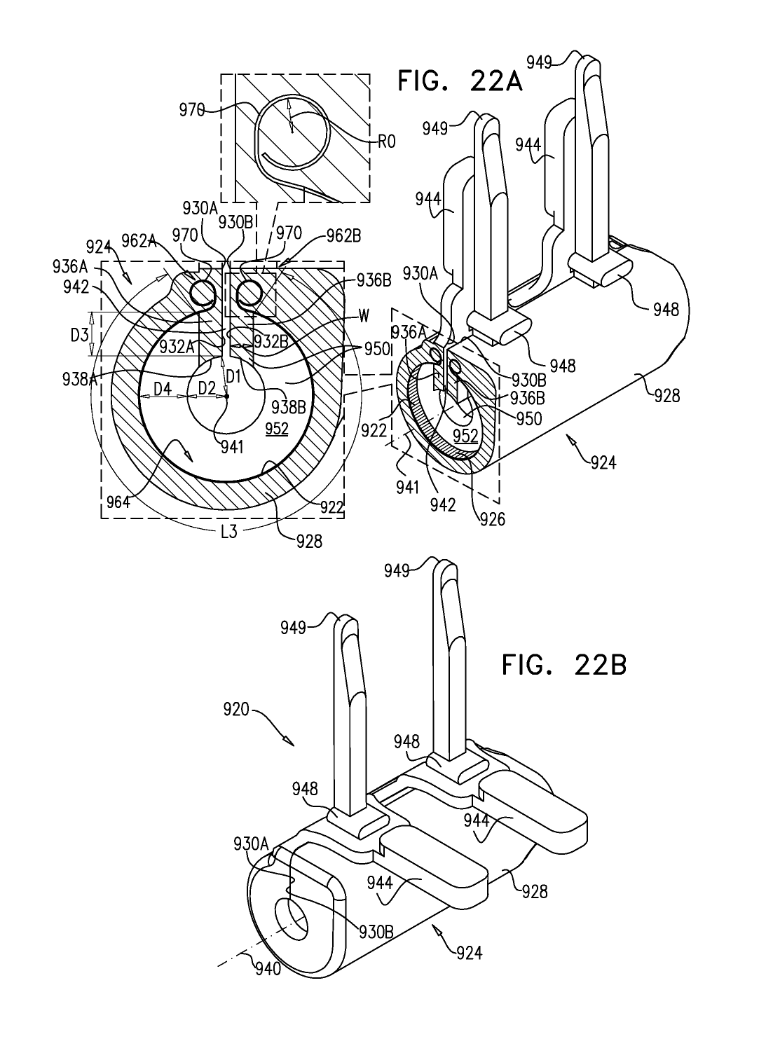

Reference is now made to FIGS. 22A and 22B, which are schematic illustrations of cuff 924 in slightly open and closed positions, respectively, in accordance with an application of the present invention. Housing 928 is shaped so as to define two edges 930A and 930B and a longitudinal slit 942 between the two edges. The slit and edges extend along an entire length of the cuff 924, typically parallel to axis 941. The housing is configured to assume:
an open position, as shown in FIG. 22A, in which two edges 930A and 930B do not touch each other; and
a closed position, as shown in FIG. 22B, in which (i) respective contact surfaces 932A and 932B of edges 930A and 930B touch each other, and (ii) the housing defines an inner surface 934 that faces and surrounds axis 941.

The cuff is placed around the tubular body tissue (such as a nerve) or elliptical cylinder (such as described above) by passing the tubular body tissue or cylinder through the slit when the housing is in the open position. The edges of the slit are brought together to bring the housing into the closed position.

Electrodes 922 are fixed to inner surface 934. Typically, electrodes 922 are shaped as partial rings, both when the housing is in the open position and when the housing is in the closed position. For some applications, electrodes 922 comprise respective strips of metal foil 960, such as described hereinbelow with reference to FIGS. 21A-C and 22A-B.

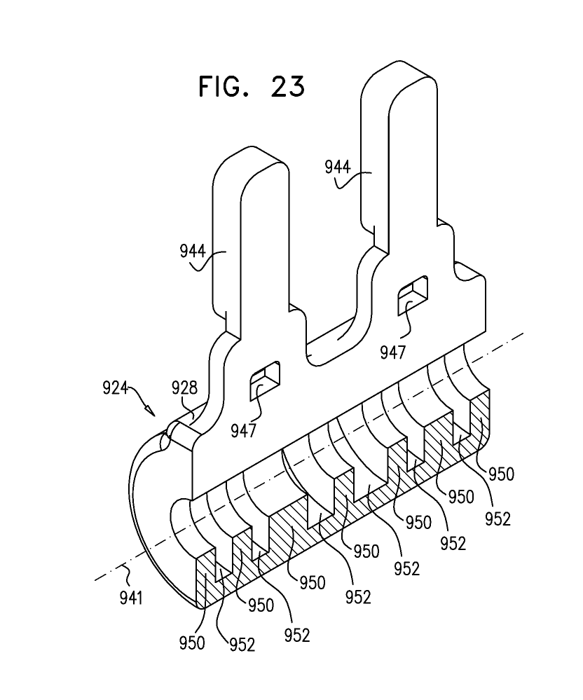

For some applications, electrode assembly 920 further comprises one or more closing elements 944, which are configured to hold edges 930A and 930B together. For some applications, each of the closing elements comprises an opening 947 near one edge 930A (labeled in FIG. 23) and a corresponding protrusion 948 on other edge 930B. To close the cuff, each of the protrusions is inserted into the corresponding slit. Optionally, each of the closing elements further comprises a tab 949, which the surgeon implanting the cuff may grasp to help pull protrusion 948 through opening 947.

Reference is again made to FIGS. 21C and 22A-B, as well as to FIG. 23, which is a schematic cut-away illustration of cuff 924, in accordance with an application of the present invention. For clarity of illustration, electrodes 922 are not shown in FIG. 23. Cuff 924 is shaped so as to define three or more annular insulating elements 950, which extend toward axis 941 from inner surface 934 of housing 928 at respective longitudinal positions along housing 928. In the exemplary configuration shown in the figures, cuff 924 is shaped so as to define seven annular insulating elements 950, two of which are disposed at respective longitudinal ends of the cuff, and five of which are disposed longitudinally within the cuff. When housing 928 is in the closed position, inner surface 934 and pairs of insulating elements 950 are shaped so as to define, at respective longitudinal positions along housing 928, respective chambers 952 open toward axis 941. (In FIGS. 22A and 22B, a longitudinal portion of cuff 924 has been cut away in order to show the inside of a longitudinal portion of one of chambers 952.) In the exemplary configuration shown in the figures, cuff 924 is shaped so as to define six annular chambers 952. For some applications, one or more of electrodes 922 are fixed within respective ones of chambers 952, typically to inner surface 934. For some applications, each of chambers 952 has a longitudinal length along the cuff of at least 0.3 mm, no more than 5 mm, and/or between 0.3 and 5 mm. For some applications, during manufacture, housing 928 and insulating elements 950 are molded as a single piece that is shaped so as to define the housing and the insulating elements. Alternatively, the insulating elements are fabricated as separate pieces and subsequently affixed to the housing.

For some applications, as best seen in FIGS. 22A and 22B, housing 928 is shaped such that contact surfaces 932A and 932B of two edges 930A and 930B extend toward axis 941 and protrude into chambers 952. Respective portions of the material of the housing define two protrusions 936A and 936B, which extend toward the axis and protrude into the chambers, and which define the portions of contact surfaces 932A and 932B, respectively, that extend toward the axis and protrude into the chambers. This configuration provides greater surface contact between contact surfaces 932A and 932B than if the contact surfaces did not extend into the chambers. This greater surface contact causes the contact surfaces to form a better electrical seal with each other, thereby reducing current leakage from the cuff.

Contact surfaces 932A and 932B extend radially inward to an average distance D1 from axis 941. For some applications, average distance D1 is at least 0.5 mm, no more than 3 mm, and/or between 0.5 and 3 mm, such as at least 1.1 mm, no more than 1.8 mm, and/or between 1.1 and 1.8 mm. Insulating elements 950 extend radially inward to an average distance D2 from axis 941. For some applications, average distance D2 is at least 0.5 mm, no more than 3 mm, and/or between 0.5 and 3 mm, such as at least 1.1 mm, no more than 1.8 mm, and/or between 1.1 and 1.8 mm. For some applications, average distance D2 is less for the insulating elements at the longitudinal ends of the cuff than for the insulating elements disposed longitudinally within the cuff. For some applications, average distance D1 equals average distance D2; in other words, insulating elements 950 and contact surfaces 932A and 932B extend to a same average distance from axis 941. For these applications, protrusions 936A and 936B define surfaces 938A and 938B, respectively, which face axis 941; surfaces 938A and 938B may have the same curvature as the surfaces of insulating elements 950 that face the axis, such as shown in FIG. 22A.

Contact surfaces 932A and 932B extend radially inward a first average distance D3 from inner surface 934 toward axis 941. For some applications, average distance D3 is at least 0.1 mm, no more than 3 mm, and/or between 0.1 and 3 mm, such as at least 0.3 mm, no more than 1.5 mm, and/or between 0.3 and 1.5 mm. Insulating elements 950 extend radially inward a second average distance D4 from inner surface 934 toward axis 941. For some applications, average distance D4 is at least 0.1 mm, no more than 3 mm, and/or between 0.1 and 3 mm, such as at least 0.3 mm, no more than 1.5 mm, and/or between 0.3 and 1.5 mm. For some applications, first average distance D3 is less than second average distance D4. Each of protrusions 36A and 36B has a width W, measured in a direction perpendicular to both (a) axis 941 and (b) contact surfaces 932A and 932B, respectively, when cuff 924 is in the closed position. For some applications, width W is at least 0.3 mm, no more than 2 mm, and/or between 0.3 and 2 mm.

Reference is again made to FIG. 21C. For some applications, an entire length L0 of cuff 924, measured along longitudinal axis 941, is at least 1 mm, no more than 40 mm, and/or between 1 and 40 mm, e.g., at least 5 mm, no more than 20 mm, and/or between 5 and 20 mm, such as 12 mm.

Reference is again made to FIGS. 21A-C and 22A-B. For some applications, at least one of electrodes 922 comprises strip of metal foil 960, which has two end portions 962A and 962B and a central portion 964 between the end portions. Central portion 964 is disposed against inner surface 934 of housing 928 such that, when the housing is in the closed position, the central portion forms a partial ring around axis 941 that defines exposed, electrically-conductive surface 926 of the central portion, which exposed surface 926 faces the axis (exposed surfaces 926 of the electrodes are shaded in the figures). (It is noted that the ring is not necessarily partially or entirely curved (as shown in the figures), and may optionally include one or more straight portions.) Typically, all of the electrodes comprise respective strips of metal foil 960.

In this configuration, at least one of end portions 962A and 962B is shaped so as to define a curved portion 970 that is embedded in and completely surrounded by the insulating material of cuff 924, thereby fixing the end portion to the insulating material. This curved portion helps firmly secure the electrode to the insulating material of the cuff by enforcing the mechanical connection between the electrode and the insulating material. Typically, curved portion 970 has an average radius of curvature R0 that is less than 10% of a length L3 of central portion 964 measured around axis 941, such as less than 5%, e.g., less than 3%. This degree of curvature helps firmly secure the electrode to the insulating material; if the curved portion were less curved, the electrode would not be as firmly secured to the insulating material. Typically, both of end portions 962A and 962B comprise respective curved portions 970 having the characteristics described herein. (As used in the present application, including in the claims, length L3 “measured around the axis” is to be understood as the length of central portion 964 measured in a direction perpendicular to axis 941, if the central portion were to be flattened, i.e., unrolled. This length may also be considered the “circumferential” length of central portion 964, even though central portion 964 typically does not form a complete loop, such as a complete ellipse or circle.)

For some applications, average radius of curvature R0 is less than 1.5 mm, e.g., less than 1 mm, such as less than 0.5 mm, and/or at least 0.2 mm, e.g., between 0.25 mm and 0.5 mm, such as 0.25 mm or 0.35 mm. Alternatively or additionally, for some applications, length L3 is at least 10 mm, no more than 20 mm, and/or between 10 mm and 20 mm, such as about 14 mm.

For some applications, as shown in the figures, strip of metal foil 960 is shaped such that a direction of curvature of curved portion 970 is opposite a general direction of curvature of central portion 964. For example, a general direction of curvature of central portion 964 as approaching end portion 962B is counterclockwise, which transitions to clockwise along curved portion 970 of end portion 962B.

Typically, curved portion 970 is shaped so as to define an arc that subtends an angle of at least 90 degrees, such as at least 180 degrees, at least 270 degrees, or at least 360 degrees. (In the figures, curved portion 970 is shows subtending an angle of slightly greater than 360 degrees, i.e., a full circle with slightly overlapping end portions.) For some applications, curved portion 970, if straightened, would have a length of at least 1.5 mm, measured in a direction perpendicular to axis 941. Alternatively or additionally, for some applications, curved portion 970, if straightened, would have a length equal to at least 5% of an entire length of the strip of metal foil 960, if straightened, which lengths are measured in a direction perpendicular to axis 941.

As mentioned above, for some applications, one or more of electrodes 922 are fixed within respective ones of chambers 952. The following tables set forth two exemplary distributions of the electrodes in the chambers. The tables also indicate, by way of example, which of the electrodes are configured by a control unit to function as cathode(s), which as anode(s), and which as passive electrode(s). Each of the passive electrodes is coupled to at least one other passive electrode, and is electrically device-coupled to neither (a) any circuitry that is electrically device-coupled to at least one cathode or at least one anode, nor (b) an energy source. The passive electrodes may be implemented using techniques described in U.S. Pat. No. 7,627,384 to Ayal et al., which is incorporated herein by reference. The chambers are numbered from left to right in FIGS. 21A and 21C.

TABLE II

| Chamber | Electrode type |
|---|---|
| 1 | Passive electrode |
| 2 | Anode |
| 3 | Cathode |
| 4 | Empty chamber (no electrode) |
| 5 | Cathode |
| 6 | Passive electrode |

TABLE III

| Segment | Electrode type |
|---|---|
| 1 | Passive electrode |
| 2 | Cathode |
| 3 | Anode |
| 4 | Empty chamber (no electrode) |
| 5 | Anode |
| 6 | Passive electrode |

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. patent application Ser. No. 10/205,475, filed Jul. 24, 2002, which issued as U.S. Pat. No. 7,778,703,
U.S. patent application Ser. No. 10/205,474, filed Jul. 24, 2002, which issued as U.S. Pat. No. 6,907,295,
International Patent Application PCT/IL03/00431, filed May 23, 2003, which published as PCT Publication WO 03/099377,
U.S. patent application Ser. No. 10/529,149, which published as US Patent Application Publication 2006/0116739,
U.S. patent application Ser. No. 10/719,659, filed Nov. 20, 2003, which issued as U.S. Pat. No. 7,778,711,
U.S. patent application Ser. No. 11/022,011, filed Dec. 22, 2004, which issued as U.S. Pat. No. 7,561,922,
U.S. patent application Ser. No. 11/234,877, filed Sep. 22, 2005, which issued as U.S. Pat. No. 7,885,709,
U.S. patent application Ser. No. 11/280,884, filed Nov. 15, 2005, which issued as U.S. Pat. No. 7,627,384,
U.S. patent application Ser. No. 12/217,930, filed Jul. 9, 2008, which published as US Patent Application Publication 2010/0010603,
U.S. patent application Ser. No. 11/347,120, filed Feb. 2, 2006, which issued as U.S. Pat. No. 7,844,346,
U.S. patent application Ser. No. 12/228,630, filed Aug. 13, 2008, which published as US Patent Application Publication 2010/0042186 and issued as U.S. Pat. No. 8,615,294,
U.S. patent application Ser. No. 12/947,608, filed Nov. 16, 2010, which published as US Patent Application Publication 2011/0098796, and/or
U.S. patent application Ser. No. 12/952,058, filed Nov. 22, 2010, which issued as U.S. Pat. No. 8,565,896.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-72. (canceled)

73. Apparatus for applying current to a nerve of a subject, comprising:
a housing, adapted to be placed in a vicinity of the nerve;
at least one cathode and at least one anode, fixed to the housing;
a passive electrode, fixed to the housing; and
a conducting element, which is electrically coupled to the passive electrode and is configured to extend to a remote location in a body of the subject at a distance of at least 1 cm from the housing.

74. The apparatus according to claim 73, wherein the distance is at least 2 cm.

75. The apparatus according to claim 74, wherein the distance is at least 3 cm.

76. The apparatus according to claim 73,
wherein the apparatus further comprises a remote electrode, and
wherein a first end of the conducting element is coupled to the passive electrode, and a second end of the conducting element is coupled to the remote electrode.

77. The apparatus according to claim 76, wherein the remote electrode is configured for insertion into muscle tissue of the subject.

78-108. (canceled)

109. A method for applying current to a nerve of a subject, comprising:
applying at least one cathodic current and at least one anodal current to the nerve; and
passively electrically coupling at least one site of the nerve to a remote location in a body of the subject at a distance of at least 1 cm from the nerve.

110. The method according to claim 109, wherein the distance is at least 2 cm.

111. The method according to claim 110, wherein the distance is at least 3 cm.

112. The method according to claim 109, wherein passively electrically coupling comprises electrically coupling a first end of a conducting element to the at least one site of the nerve, and inserting a second end of the conducting element into muscle tissue of the subject.

113-155. (canceled)
